(19) United States
(12) Patent Application Publication
(10) Pub. No.: US 2011/0286947 A1
(43) Pub. Date: Nov. 24, 2011

(54) METHOD FOR MAKING UP OR CARING FOR KERATINOUS SUBSTANCES WITH NONCROSSLINKED POLYROTAXANES

(75) Inventors: Timo Luukas, Massy (FR); Nathalie Jager Lezer, Verrieres-Le-Buisson (FR); Pascal Arnaud, L'hay Les Roses (FR)

(73) Assignee: L'OREAL, Paris (FR)

(21) Appl. No.: 12/376,420

(22) PCT Filed: Aug. 3, 2007
(86) PCT No.: PCT/EP07/58067

§ 371 Date: Jun. 3, 2009

(30) Foreign Application Priority Data
Aug. 4, 2006 (FR) 0653297

(51) Int. Cl. A61K 8/02; A61K 8/88; A61K 8/895; A61Q 3/02; A61Q 1/04; A61Q 1/12; A61Q 1/10; A61K 8/73; A61K 8/81

(52) U.S. Cl. 424/61; 424/70.1; 424/70.13; 424/70.17; 424/70.12; 424/70.16; 424/70.15; 424/64; 424/69; 424/70.7

(57) ABSTRACT

A subject-matter of the invention is a cosmetic method for making up or for the nontherapeutic care of keratinous substances chosen from the nails, skin, lips or eyelashes, the method consisting in: a. depositing on the said keratinous substances at least one layer of at least one first composition comprising at least one first noncrosslinked polyrotaxane and at least one second noncrosslinked polyrotaxane, b. subjecting the said composition, simultaneously with or subsequent to its application, to at least one chemical, physicochemical and/or mechanical stimulus.

A subject-matter of the present invention is a method for making up or caring for keratinous substances which consist in applying to the said keratinous substances, at least one noncrosslinked polyrotaxane and at least one second noncrosslinked polyrotaxane which crosslink on the said keratinous substances.

The compositions according to the invention can be compositions for making up or caring for keratinous substances, in particular the skin, nails, lips and keratinous fibres, especially the eyelashes, and preferably makeup compositions.

Each composition can be a free or compacted powder, a foundation, a face powder, an eyeshadow, a concealer, a blusher, a lipstick, a lip balm, a lip gloss, a lip pencil, an eye pencil, a mascara, an eyeliner, a nail varnish or also a product for making up the body or for colouring the skin.

The care composition can be a product for caring for the eyelashes, nails or lips, for caring for the skin of the body and face, in particular an antisun product, or a product for colouring the skin (such as a self-tanning product).

Consumers are looking for cosmetic products which make it possible to obtain an increase in the perception of the volume of the keratinous substances which they desire to make up. In particular, a volumizing or body-bestowing effect on the eyelashes is desired for mascaras, a fullness effect is desired for glosses and lipsticks, and properties of modelling the face and of masking imperfections of the skin (wrinkles, fine lines, defects of pigmentation, loss in colour of the lips, rosacea) are required by users of foundations and lipsticks.

In addition, these cosmetic compositions must exhibit good hold over time, so that the aesthetic effect obtained is maintained.

Currently, the remodelling and the increase in volume of certain parts of the face or body is obtained by injection of substances, such as silicone gels. This type of remodelling is generally carried out under local anaesthesia. In addition, this type of remodelling is lengthy, tedious and expensive.

Furthermore, it is known that an effective volume can be produced by applying a light tint and a dark tint side by side, the light tint being applied to the area which it is desired to enhance. To produce this effect conventionally requires the use of two different compositions and depends on the fitness of the one who is applying them. This technique is furthermore difficult to employ in making up the lips.

The use is also known, for example from the documents EP 0 953 330, WO 01/51015 or EP 1 382 323, of optical effect pigments (goniochromatic or interference pigments) for modifying the perception of the volume of the part of the body to which the composition is applied, according to the angle of observation or the angle of incidence of the light.

In the case of mascaras, use is generally made of compositions having a high solids content, in order to contribute material to the keratinous fibres and thus to obtain a makeup result in which volume or loading are more or less bestowed.

Nevertheless, the increase in the solids content of solids such as waxes, fillers or pigments leads to an increase in the consistency of the product obtained and thus to an application to fibres which is problematic and difficult as the composition is thick and viscous, gives a granular and nonsmooth appearance to the deposited layer and is deposited with difficulty, in heterogeneous fashion and in clusters.

A description is also given in the document EP 1 525 876 of the use in a mascara of a polymer capable of swelling under the action of heat.

The documents US 6045783 and EP 1 195 157 also describe cosmetic compositions comprising polymers which are superabsorbent with regard to water.

The aim of the present invention is to provide a novel route for the formulation of cosmetic compositions capable of crosslinking on keratinous substances and of generating, on the said keratinous substances, a body-bestowing deposited layer having good properties of hold over time and a comfortable deposition on the skin, lips or eyelashes.

The inventors have discovered that it is possible to obtain such properties by using a system comprising compounds which crosslink in situ, so as to adhere better to keratinous substances. In addition, once applied to keratinous substances, these compounds absorb water, thus bringing about an increase in the volume of the deposited layer. The keratinous substances thus give the impression of being thicker, fuller or smoother by filling in their rough edges.

More specifically, a subject-matter of the invention is a cosmetic method for making up or for the nontherapeutic care of keratinous substances chosen from the nails, skin, lips or eyelashes, the method consisting in:
a. depositing on the said keratinous substances at least one layer of at least one first composition comprising at least one first noncrosslinked polyrotaxane and at least one second noncrosslinked polyrotaxane,
b. subjecting the said composition, simultaneously with or subsequent to its application, to at least one chemical, physicochemical and/or mechanical stimulus.

This first noncrosslinked polyrotaxane and this second noncrosslinked polyrotaxane, which are identical or different, are capable of polymerizing when they are subjected to a stimulus, that is to say an action, for example chemical, physicochemical or mechanical action, exerted on the composition or compositions comprising them.

According to one embodiment, the stimulus comprises at least one crosslinking agent. This is why, according to one alternative form, a subject-matter of the invention is a cosmetic method for making up or for the nontherapeutic care of keratinous substances chosen from the nails, skin, lips or eyelashes, the method consisting in depositing, on the said keratinous substances:
a. at least one layer of a first composition comprising at least one first noncrosslinked polyrotaxane,
b. at least one layer of a second composition comprising a crosslinking agent,
   the first composition and/or the second composition comprising at least one second noncrosslinked polyrotaxane.

Preferably, the second noncrosslinked polyrotaxane is present in the first composition.

According to another embodiment, a crosslinking agent is grafted to the first and/or to the second polyrotaxane.

This is why a further subject-matter of the invention is a cosmetic method for making up or for the nontherapeutic care of keratinous substances chosen from the nails, skin, lips or eyelashes, the method consisting in depositing, on the said keratinous substances, at least one layer of a first composition comprising at least one first noncrosslinked polyrotaxane and at least one second noncrosslinked polyrotaxane, the first noncrosslinked polyrotaxane and/or the second noncrosslinked polyrotaxane being grafted with a crosslinking agent.

A further subject-matter of the invention is a cosmetic method for making up or for the nontherapeutic care of keratinous substances chosen from the nails, skin, lips or eyelashes, the method consisting in depositing, on the said keratinous substances:
at least one layer of a first composition comprising at least one first noncrosslinked polyrotaxane,
at least one layer of a second composition comprising at least one second noncrosslinked polyrotaxane,
   at least one of the first and/or second noncrosslinked polyrotaxanes being grafted with a crosslinking agent.

The terms first and second compositions do not in any way condition the order of application of the said compositions to the keratinous substances. The second composition can be applied to the first composition and vice versa.

According to one embodiment, at least one layer of the first composition is applied to the keratinous substances and then at least one layer of the second composition is applied to all or part of the first layer.

Several layers of each first and second composition can also be applied alternately to the keratinous substances.

According to another alternative form, a further subject-matter of the invention is a cosmetic method for making up or for the nontherapeutic care of keratinous substances chosen from the nails, skin, lips and eyelashes, consisting in:
a. mixing, at the time of use:
at least one first composition comprising a cosmetically acceptable medium and at least one first noncrosslinked polyrotaxane, and
at least one second composition comprising a crosslinking agent,
   the first composition and/or the second composition comprising at least one second noncrosslinked polyrotaxane,

then
b. applying at least one layer of the said mixture to the nails, skin, lips or eyelashes.

According to another aspect, another subject-matter of the present invention is a kit for making up or for the nontherapeutic care of keratinous substances chosen from the nails, skin, lips and eyelashes, comprising:
i) at least one first composition comprising at least one first noncrosslinked polyrotaxane,
ii) at least one second composition comprising a crosslinking agent,
   the first composition and/or the second composition comprising at least one second noncrosslinked polyrotaxane.

Preferably, the makeup kit according to the invention comprises the first and second compositions in separate packaging.

Each composition can be packaged separately in the same article of packaging, for example in a two-compartment pen, the base composition being delivered via one end of the pen and the top composition being delivered via the other end of the pen, each end being closed in particular in leaktight fashion by a cap.

Alternatively, each of the compositions can be packaged in a different article of packaging.

After bringing into contact, the first and second polyrotaxanes crosslink together and a deposited layer possessing good hold is obtained on the keratinous substances; this deposited layer is capable of increasing in volume by formation of a gel in the presence of a fluid. This fluid, preferably hydrophilic, can, for example, be sweat, saliva, tears, residual water of the skin, lips, nails and/or eyelashes, ambient moisture or any other natural or artificial liquid. It can be contributed by an external source, for example by moistening the keratinous substances before or after application of the compositions (for example with a spray, natural or artificial tears). The fluid can also be a polar solvent, such as, for example, propylene glycol or ethylene glycol.

The fluid, in particular water, can also be added directly to the composition or compositions comprising the noncrosslinked polyrotaxane or polyrotaxanes before application.

According to an embodiment, the fluid can be contributed via at least one additional layer of at least one third composition comprising an aqueous medium which is applied to the layer or layers of first and/or second composition in order to bring about the swelling of the crosslinked polyrotaxane.

These compositions, the volume of which is capable of increasing, make it possible to lastingly conceal defects of appearance of keratinous substances (blemishes, shadows under the eyes, folds, hollows, thinness) and may possibly confer an increased volume on the nails, skin, eyelashes or lips.

They make it possible to obtain, on keratinous substances, a deposited layer exhibiting good hold and satisfactory mechanical or rheological properties, such as a pleasant texture suitable for the use of the composition and a satisfactory elasticity, making it possible to obtain a comfortable deposited layer on the keratinous substances.

In addition, it is possible to incorporate a colouring material or an active principle in the first and/or the second composition, which makes it possible to efficiently trap said active principle or said colouring material in the gel which is formed on the keratinous substances after swelling of the deposited layer.

Furthermore, due to their water absorption capacity and thus due to sweat, these compositions can have a particular application in the field of foundations or mattifying creams. In addition, the water-swollen deposited layer (gel) makes it possible to prevent dehydration of the skin and feelings of discomfort and of tightness.

According to one embodiment, the deposited layer obtained on the keratinous substances after application of the first and/or second compositions and then swelling with water is subjected to a heat source. The heating then brings about the partial or complete evaporation of the water present in the gel and the retraction of the film on the keratinous substances and thus a tensioning effect of the film. It is thus possible to obtain, in the case of mascaras, a curving effect on the eyelashes or, in the case of products for caring for or making up the skin, an effect of smoothing the skin and of reducing wrinkles and fine lines.

NonCROSSLINKED POLYROTAXANES

Polyrotaxanes form part of the chemical family of the inclusion compounds, which comprise a first molecular entity which forms a cavity of limited size in which is housed a molecular entity of a second chemical type.

JP09216815 of Noevir Co. Ltd (1997) and JP09315937 of Shiseido Co. Ltd (1997) have described cosmetic products comprising pseudopolyrotaxanes.

The term “pseudopolyrotaxane” is understood to mean a supramolecular edifice which comprises at least one linear molecule and at least two cyclic molecules strung along the said linear molecule, the linear molecule and the cyclic molecules not being bonded via covalent bonds, with the result that the cyclic molecules can move freely along the linear molecule.

The molecules described in these documents comprise a backbone on which cyclic molecules (cyclodextrins) are included. However, the compositions do not increase in volume sufficiently once applied to keratinous substances and their hold over time is poor. In addition, the cyclic molecules have a tendency to become unstrung when the pseudopolyrotaxane is dissolved.

A “polyrotaxane” is obtained from a pseudopolyrotaxane, to which is attached, at each end of the linear molecule, a “blocking” molecular structure which prevents the cyclic molecules and the linear molecule from separating, if appropriate.

The composition or compositions employed in the process according to the invention comprise at least one first noncrosslinked polyrotaxane and at least one second noncrosslinked polyrotaxane, that is to say compounds which are not bonded to one another and which, subjected to a stimulus, are capable of crosslinking with one another, by formation of at least one bond, which can be chemical or physical, between a cyclic molecule of the first polyrotaxane and at least one cyclic molecule of the second polyrotaxane, to form a crosslinked polyrotaxane.

The bond can in particular be a metallic bond, an ionic bond, a covalent bond, an interaction resulting from the formation of charge transfer complexes, a weak interaction of hydrogen bond, Van der Waal's bond or π-π bond type, or a mixture of these.

A polyrotaxane is thus a supramolecular assemblage in which cyclic molecules are “included” by a linear molecule. To prevent the cyclic molecules from becoming unstrung from the linear molecule, the ends of the linear molecule are functionalized by bulky or ionic groups (blocking molecular structures).

a) Linear Molecules

In the present invention, the expression “linear molecule” is intended to denote a substantially “linear” molecule. This means that a linear molecule can comprise one or more branch chains, provided that the cyclic molecules can be rotated about or moved along the linear molecule.

The length of the “linear” molecule is not limited to a specific length, provided that the linear molecule allows the cyclic molecules to turn round on themselves or to move along the said linear molecule.

The linear molecule of the first polyrotaxane and/or the linear molecule of the second polyrotaxane can be chosen independently of one another from polymers, in particular:
hydrophilic polymers, such as a poly(vinyl alcohol), a polyvinylpyrrolidone, a poly((meth)acrylic acid), polymers derived from cellulose (carboxymethyl-cellulose, hydroxyethylcellulose, hydroxypropyl-cellulose and the like), a polyacrylamide, polyalkylene glycols, such as polyethylene glycols and polypropylene glycols, polytetrahydrofurans, poly(vinyl acetal)s, a poly(vinyl methyl ether), polyamines, polyethylene-imine, casein, gelatin, starch, and their copolymers;
hydrophobic polymers, for example polyolefins, such as polyethylenes, polypropylenes, polyisoprenes, polyisobutylenes or polybutadienes; copolymers of olefins, such as ethylene/butylene copolymers; polyesters, polydimethylsiloxanes, poly(vinyl chloride), polystyrene, acrylonitrile/styrene copolymers, polymers and copolymers of (meth)acrylic esters, such as poly(methyl methacrylate) or acrylonitrile/methyl acrylate copolymers; polycarbonates, polyurethanes, vinyl chloride/vinyl acetate copolymers or poly(vinyl butyral); and their derivatives.

Preference is given, among these compounds, to polyethylene glycols, polyisoprenes, polyisobutylenes, polybutadienes, polypropylene glycols, polytetrahydrofurans, polydimethylsiloxanes, polyethylenes and polypropylenes. Polyethylene glycols are particularly preferred.

The linear molecules advantageously have, independently of one another, a weight-average molecular weight of greater than or equal to 350 g/mol, for example ranging from 350 to 2 000 000, preferably ranging from 1500 to 1 000 000, more preferably ranging from 2800 to 800 000, even better still ranging from 7000 to 700 000, for example ranging from 10 000 to 600 000 or from 10 000 to 500 000.

The linear molecules preferably carry reactive groups at each end. The fact of carrying the reactive groups makes it possible to facilitate the reaction with the molecular structures intended to prevent separation between the linear molecules and the cyclic molecules which they carry.

The reactive groups depend on the blocking molecular structures to be employed.

Mention may be made, as examples, of hydroxyl groups, amino groups, tosylate groups, polymerizable groups, activated ester groups, such as N-hydroxysuccinimide ester groups, carboxyl groups, thiol groups and the like.

b) Cyclic Molecules

In the present invention, a “cyclic molecule” denotes a molecule comprising at least one cyclic structure. The cyclic molecule can comprise two or more cyclic structures or a double ring. The cyclic molecule can be a macrocycle, such as a cyclodextrin.

The cyclic molecules of the first and second polyrotaxanes can be chosen, independently of one another, from:
cyclodextrins, for example cc-cyclodextrin, β-cyclodextrin, γ-cyclodextrin, dimethylcyclodextrin and glucosylcyclodextrin, and their derivatives,
crown ethers,
benzocrown ethers, dibenzocrown ethers and dicyclohexanocrown ethers, and their derivatives.

The size of the internal cavity or cavities of the cyclic molecules can vary according to the linear molecule chosen. In any case, cyclic molecules are chosen which can be strung along the linear molecule. Thus, the cavity of the cyclic molecule will preferably have a diameter greater than the diameter of the cross section of a minimum imaginary cylinder in which the linear molecule can be included.

Preference is given, among the cyclic molecules which can be used, to cyclodextrins.

According to one embodiment, α-cyclodextrin is used as cyclic molecule and a polyethylene glycol is used as linear molecule.

The cyclic molecules preferably have groups capable of generating bonds which are not situated in their cavity. This makes it possible to subsequently bond the cyclic molecules to one another via a chemical or physical bond. The reactive groups of the cyclic molecules can comprise, for example, hydroxyl, amino, carboxyl or thiol groups. Furthermore, it is preferable to choose cyclic molecules having reactive groups which do not react with the blocking structures during the blocking reaction between the said blocking structures and the linear molecules.

The ratio of the number of cyclic molecules strung along a linear molecule to the maximum amount of cyclic molecules of the same nature which could be strung along this linear molecule ranges from 0.001 to 0.6, preferably from 0.01 to 0.5 and better still from 0.05 to 0.4. This ratio may be referred to as “inclusion amount”.

The maximum inclusion amount is standardized as being equal to 1. It corresponds to the amount at which a linear molecule makes it possible to include a maximum of cyclic molecules.

It is preferable for the linear molecule not to exhibit a dense stack of cyclic molecules. This dense stack state corresponding to the maximum inclusion amount equal to 1. The fact of creating a non-dense stack of cyclic molecules makes it possible to retain molecular segments which can be moved, with the result that the crosslinked polyrotaxane exhibits a high fracture strength, a high entropic elasticity, a superior expandability and/or a superior restoring property, and, if desired, a high absorbability or a high hygroscopicity.

According to one embodiment, the cyclic molecules can be cyclized after inclusion of the linear molecules. More specifically, it is possible to use a precursor of the cyclic molecules having at least one open segment analogous to the letter “C”.

In this case, the “C” segments can be closed after the inclusion of the linear molecule or after the blocking of the linear molecule with a blocking group. For the molecules having a segment analogous to the letter “C”, see M. Asakawa et al., Angewandte Chemie International, 37(3), 333-337 (1998), and M. Asakawa et al., European Journal of Organic Chemistry, 5, 985-994 (1999), both being incorporated here by way of reference.

c) Molecular Structures Situated at the Chain End of the Linear Molecules: Blocking Structures

The blocking structures have to keep the cyclic molecules strung along the linear molecule.

These blocking structures can prevent the cyclic molecules from separating from the linear molecule due to their high steric volume.

The blocking structures situated at each end of each linear molecule can also prevent the cyclic molecules from decomplexing from the linear molecule by exhibiting specific ionic charges.

The expression “molecular structure” denotes here a molecule, a macromolecule or a solid support.

A macromolecule or a solid support can include several blocking sites. A blocking structure of a macromolecule can be present in the main chain or in a side chain.

When a blocking structure is a macromolecule A, the macromolecule A can constitute a matrix, a portion of which comprises pseudopolyrotaxanes, or conversely the pseudopolyrotaxane can constitute a matrix, a portion of which comprises the macromolecule A.

The blocking molecular structures can be chosen from:
dinitrophenyl groups, such as the 2,4- and 3,5-dinitrophenyl groups;
cyclodextrins;
adamantane groups;
trityl groups;
fluoresceins;
pyrenes;
naphthalimides; and
their combinations.

According to one embodiment, when the linear molecule is a polyethylene glycol, the cyclic molecules can be chosen from α-cyclodextrin, dinitrophenyl groups, such as the 2,4- and 3,5-dinitrophenyl groups, adamantane groups, trityl groups, fluoresceins, pyrenes and their combinations.

d) Crosslinking

The first and/or second polyrotaxanes are capable of crosslinking by formation of at least one chemical bond or of at least one physical bond (preferably at least two physical bonds) between at least one cyclic molecule of the noncrosslinked polyrotaxanes, when they are subjected to a stimulus.

As set out above, the stimulus can be an action, for example a chemical, physicochemical or mechanical action, exerted on the first composition and/or the second composition.

The crosslinking can thus be carried out thermally, photochemically, chemically and/or mechanically, in the presence or absence of a crosslinking agent.

It can, for example, be carried out at the temperature of the skin or by using means not specifically intended for heating, such as a hot body (cup or a hot drink). The composition can also be heated using a means specifically dedicated to heating, such as, for example, a means which propels hot air, such as a hairdryer, or a heating device, such as, for example, a heating applicator.

According to one embodiment, the first and second polyrotaxanes crosslink via a chemical or physical bond which can be formed by a simple bond or by a bond involving different atoms or molecules. The said bond can be obtained by reaction of the said two cyclic molecules with a crosslinking agent or a photocrosslinking agent.

The term “crosslinking agent” is understood to mean a compound capable of creating at least one chemical bond (covalent bond) or physical bond (ionic bond, hydrogen bond, π-π interactions or Van der Waals forces) between two or more molecules.

A cyclic molecule preferably has one or more reactive groups on the outside of the nucleus, as described above. In particular, it is preferable, after the formation of a blocked polyrotaxane molecule, for some cyclic molecules of different polyrotaxanes to be crosslinked to one another by means of a crosslinking agent. This reaction can be carried out under the action of temperature or of a variation in pH. In this case, the conditions of the crosslinking reaction must be conditions under which the blocking groups of the blocked polyrotaxane are not removed.

According to one embodiment, the first composition and/or the second composition comprises at least one crossing agent, alone or in combination with the noncrosslinked polyrotaxane or polyrotaxanes.

According to an alternative form, the crosslinking agent can be grafted to a filler or to a colouring material, such as those described later.

According to one embodiment, the crosslinking agent is grafted to the first and/or second noncrosslinked polyrotaxane, in particular to the cyclic segment of the said noncrosslinked polyrotaxane or polyrotaxanes.

According to one embodiment, the first composition or the second composition additionally comprises at least one crosslinking agent.

Preferably, the composition comprising a crosslinking agent does not comprise noncrosslinked polyrotaxane; in particular, the first composition employed in the process according to the invention does not comprise crosslinking agent.

Use may be made, as crosslinking agents, of crosslinking agents well known in the prior art.

Use may be made, for example, of:
i) compounds having at least two polymerizable double bonds, such as, for example
   di- or polyvinyl compounds, such as divinylbenzene, divinyltoluene, divinylxylene, divinyl ether, divinyl ketone, divinyl sulphone or trivinylbenzene,
   di- or polyesters resulting from the reaction of unsaturated mono- or polycarboxylic acids with polyols, such as di- or triesters of (meth)acrylic acids and of polyols chosen, for example, from ethylene glycol, trimethylolpropane, glycerol, polyoxyethylene glycols, polyoxypropylene glycols and their mixtures; polyesters resulting from the reaction of any one of the abovementioned polyols with an unsaturated (or ethylenic) carboxylic acid, such as, for example, maleic acid; esters resulting from the reaction of a polyepoxide with (meth)acrylic acid,
   bis(meth)acrylamides, such as N,N-methylenebisacrylamide,
   “carbamyl” esters resulting from the reaction of polyisocyanates (such as tolylene diisocyanate, hexamethylene diisocyanate, 4,4′-diphenylmethane diisocyanate, phenylene diisocyanates, 1,1′-carbonyldiimidazole or prepolymers comprising an NCO group obtained by reacting such diisocyanates with compounds comprising active hydrogen atoms) with monomers comprising hydroxyl groups; mention may in particular be made of the dicarbamyl esters of (meth)acrylic acids obtained, for example, by reaction of the abovementioned diisocyanates with hydroxyethyl (meth) acrylates,
   di- or poly(meth)allyl ethers or polyols, such as alkylene glycols, glycerol, polyalkylene glycols, polyoxyalkylene polyols or carbohydrates, such as polyethylene glycol diallyl ether, allylated starch and allylated cellulose,
   di- or polyallyl esters of polycarboxylic acids, such as diallyl phthalate or diallyl adipate,
   esters of unsaturated mono- or polycarboxylic acids with mono(meth)allyl ethers of polyols, such as the (meth)acrylic acid ester of the monoallyl ether of polyethylene glycol.
ii) compounds having at least one polymerizable double bond and at least one functional group which reacts with the noncrosslinked polyrotaxane or polyrotaxanes, in particular unsaturated ethylenic compounds comprising at least one group which reacts with carboxyl, carboxylic anhydride, hydroxyl; amine or amide groups. Mention may be made, for example, of N-methylol(meth)acrylamide, glycidyl (meth) acrylate, and their mixtures.
iii) compounds having at least two functional groups which react with the noncrosslinked polyrotaxane or polyrotaxanes, which are in particular di- or polyfunctional compounds comprising groups which react with carboxyl, carboxylic anhydride, hydroxyl, amine or amide groups. Mention may be made of glyoxal, polycarboxylic acids, such as phthalic acid or adipic acid, polyols such as ethylene glycol, polyamines, such as alkylenediamines (for example, ethylenediamine) or polyalkylenepolyamines, bis- or polyepoxides, such as glycidyl epoxides, cycloaliphatic epoxides (for example, 3,4-epoxycyclohexylmethyl 3,4-epoxycyclohexanecarboxylate), epoxidized vegetable oils, such as epoxidized soybean or linseed oils, polyethylene oxide-succinimidyl glutarates, such as polyethylene oxide-tetrasuccinimidyl glutarate, bisepoxybutane or glycidyl ethers, such as bisphenol A diglycidyl ether, bishydrazides, tris- or polyhydrazides, carbodiimides, dialdehydes (for example, PEG-dialdehydes), and their mixtures.

Mention may be made, as other examples of crosslinking agents, of cyanuric chloride, trimesoyl chloride, terephthaloyl chloride, epichlorohydrin, dibromobenzene, glutaraldehyde, bis(acid chlorides) (for example, sebacoyl dichloride), tri(acid chlorides) and the like.

The crosslinking agent can be chosen from coupling agents of silane type (for example, alkoxysilanes) and/or titanium-based coupling agents (for example alkoxytitanium compounds).

Use may also be made, as crosslinking agents, of crosslinking agents capable of forming, between them, at least two physical bonds (in particular hydrogen bonds), it being possible for these crosslinking agents to be, independently of one another, carried by a noncrosslinked polyrotaxane or present in either of the first and/or second compositions. Mention may be made, as examples of such agents, of the 2,6-diaminopyridine and uracils pair, the barbituric acid and triaminopyridines pair, or also ureidopyrimidinones, ureidotriazines or cyanuric acid derivatives which crosslink with one another.

Mention may be made, as crosslinking agent capable of establishing one or more ionic bonds, of polyvalent metallic compounds which form ionic crosslinkings, such as, for example, oxides, hydroxides and weak acid salts (for example, carbonates, acetates, and the like) of alkaline earth metals (for example, calcium or magnesium), of zinc or of aluminium; mention may be made, for example, of calcium oxide, zinc diacetate or aluminium sulphate.

Such crosslinking agents (also known under the name of “synthons”) are described, for example, in the following references: “Supramolecular Polymers”, L. Brunsveld, B. J. B. Folmer, E. W. Meijer and R. P. Sijbesma, Chemical Reviews, 2001, 4071-4098 or Supramolecular Chemistry, J M Lehn, VCH, 1995.

Mention may be made, as other examples, of various photocrosslinking agents which are employed for materials designed for soft contact lenses, for example photocrosslinking agents based on stilbazolium salts, such as formylstyrylpyridinium salts (see K. Ichimura et al., Journal of Polymer Science, edition on the chemistry of polymers, 20, 1411-1432 (1982), incorporated here by way of reference), and other photocrosslinking agents, for example photocrosslinking agents by photodimerization, specifically cinnamic acid, anthracene, thymines and the like.

The crosslinking agents preferably have weight-average molecular weights of less than 2000, preferably of less than 1000, better still of less than 600 and very particularly of less than 400.

In the case where a-cyclodextrin is used as cyclic molecule and where a crosslinking agent is used to crosslink it, mention may be made, as examples of crosslinking agent, of cyanuric chloride, tolylene 2,4-diisocyanate, 1,1′-carbonyldiimidazole, trimesoyl chloride, terephthaloyl chloride, alkoxysilanes, such as tetramethoxysilane and tetraethoxysilane, cycloaliphatic epoxides, such as 3,4-epoxycyclohexylmethyl 3,4-epoxycyclohexanecarboxylate, polyethylene oxide-succinimidyl glutarates, such as polyethylene oxide-tetrasuccinimidyl glutarate, bishydrazides, and the like, and their mixtures. In particular, it is preferable to use a-cyclodextrin as cyclic molecule and cyanuric chloride as crosslinking agent.

Preparation of a Noncrosslinked Polyrotaxane

The compounds according to the present invention can be prepared according to the teaching of Patent Application EP 1 283 218, with the exception of the crosslinking stage.

First of all, the cyclic molecules and the linear molecules are mixed in order to prepare the pseudopolyrotaxanes, in which the cyclic molecules are strung along the linear molecules. Secondly, the polyrotaxanes are prepared by blocking each end of the linear molecules with blocking groups, so as to prevent the removal of the cyclic molecules.

According to one embodiment of the invention, α-cyclo-dextrin is used as cyclic molecule, a polyethylene glycol is used as linear molecule, a 2,4-dinitrophenyl group is used as blocking group and cyanuric chloride is used as crosslinking agent.

First of all, each end of the polyethylene glycol is converted to an amino group, in order to be able subsequently to attach a blocking group to the end of the polyethylene glycol and to form the polyrotaxane. In an alternative form, use may be made of diamine-terminated PEG/PPO copolymers, sold by Huntsman under the Jeffamine reference.

Subsequently, the a-cyclodextrin and the aminated polyethylene glycol derivative are mixed in order to prepare the pseudopolyrotaxane. The duration of the mixing ranges from 1 to 48 hours and the mixing temperature ranges from 0 to 100° C., so that the inclusion amount of α-cyclodextrin with regard to the polyethylene glycol derivative ranges from 0.001 to 0.6.

Generally, a polyethylene glycol having an average molecular weight of 20 000 makes it possible to include at most 230 α-cyclodextrin molecules. The maximum inclusion amount, corresponding to 230 molecules, is equal to 1.

According to one embodiment, 60 to 65 (63) α-cyclodextrin molecules are on average strung over one polyethylene glycol molecule, which corresponds to a degree of inclusion ranging from 0.26 to 0.29 (0.28) with respect to the maximum inclusion amount. The α-cyclodextrin inclusion amount can be determined by NMR, light absorption or elemental analysis.

The pseudopolyrotaxane obtained is reacted with 2,4-dinitrofluorobenzene dissolved in DMF, which makes it possible to obtain the noncrosslinked polyrotaxane.

The polyrotaxane can be used as is or partially or completely prehydrated.

It is possible, for example, to dissolve the noncrosslinked polyrotaxane beforehand in a basic aqueous solution, for example a sodium hydroxide solution.

The first and second noncrosslinked polyrotaxanes can be present in a content ranging from 0.1 to 80% by weight, preferably from 1 to 50% by weight and more preferably from 2 to 30% by weight, with respect to the total weight of each first or second composition.

According to a specific embodiment, the first and/or second composition according to the invention comprises, in addition to the first noncrosslinked polyrotaxane and/or the second noncrosslinked polyrotaxane, at least one crosslinked polyrotaxane which has been obtained by crosslinking, prior to its introduction into the composition, at least one first noncrosslinked polyrotaxane and at least one second noncrosslinked polyrotaxane, as described above.

Liquid Fatty Phase

The first composition and/or the second composition advantageously comprises a liquid fatty phase.

The term “liquid fatty phase” is understood to mean, within the meaning of the patent application, a fatty phase which is liquid at ambient temperature (25° C.) and atmospheric pressure (760 mmHg) and which is composed of one or more nonaqueous fatty substances which are liquid at ambient temperature, also known as oils or organic solvents.

The oil can be chosen from volatile oils and/or non-volatile oils, and their mixtures.

The oil or oils can be present in the composition according to the invention in a content ranging from 1% to 80% by weight, preferably from 5% to 50% by weight, with respect to the total weight of the composition.

The term “volatile oil” is understood to mean, within the meaning of the invention, an oil capable of evaporating on contact with keratinous substances in less than one hour at ambient temperature and atmospheric pressure. The volatile organic solvent or solvents and the volatile oils of the invention are volatile cosmetic organic solvents and oils which are liquid at ambient temperature and which have a nonzero vapour pressure, at ambient temperature and atmospheric pressure, ranging in particular from 0.13 Pa to 40 000 Pa (10⁻³ to 300 mmHg), in particular ranging from 1.3 Pa to 13 000 Pa (0.01 to 100 mmHg) and more particularly ranging from 1.3 Pa to 1300 Pa (0.01 to 10 mmHg).

The term “nonvolatile oil” is understood to mean an oil which remains on keratinous substances at ambient temperature and atmospheric pressure for at least several hours and which has in particular a vapour pressure of less than 10⁻³ mmHg (0.13 Pa).

These oils can be hydrocarbon oils, silicone oils, fluorinated oils or their mixtures.

The term “hydrocarbon oil” is understood to mean an oil comprising mainly hydrogen and carbon atoms and optionally oxygen, nitrogen, sulphur and phosphorus atoms. Volatile hydrocarbon oils can be chosen from hydrocarbon oils having from 8 to 16 carbon atoms, in particular branched C₈-C₁₆ alkanes, such as C₈-C₁₆ isoalkanes of petroleum origin (also known as isoparaffins), such as isododecane (also known as 2,2,4,4,6-pentamethylheptane), isodecane or isohexadecane, for example the oils sold under the Isopar or Permethyl tradenames, branched C₈-C₁₆ esters, isohexyl neopentanoate, and their mixtures. Other volatile hydrocarbon oils, such as petroleum distillates, in particular those sold under the Shell Solt name by Shell, can also be used. Preferably, the volatile solvent is chosen from volatile hydrocarbon oils having from 8 to 16 carbon atoms and their mixtures.

Use may also be made, as volatile oils, of volatile silicones, such as, for example, volatile linear or cyclic silicone oils, in particular those having a viscosity ≦8 centistokes (8×10⁻⁶ m²/s) and having in particular from 2 to 7 silicon atoms, these silicones optionally comprising alkyl or alkoxy groups having from 1 to 10 carbon atoms. Mention may in particular be made, as volatile silicone oil which can be used in the invention, of octamethylcyclotetrasiloxane, decamethylcyclopentasiloxane, dodecamethylcyclohexabiloxane, heptamethylhexyltrisiloxane, heptamethyloctyltrisiloxane, hexamethyldisiloxane, octamethyltrisiloxane, decamethyltetrasiloxane, dodecamethylpentasiloxane and their mixtures.

Mention may also be made of the volatile linear alkyltrisiloxane oils of general formula (I):

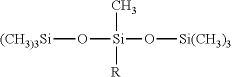

where R represents an alkyl group comprising from 2 to 4 carbon atoms, one or more hydrogen atoms of which can be substituted by a fluorine or chlorine atom.

Mention may be made, among the oils of general formula (I), of:

3-butyl-1,1,1,3,5,5,5-heptamethyltrisiloxane,

3-propyl-1,1,1,3,5,5,5-heptamethyltrisiloxane, and

3-ethyl-1,1,1,3,5,5,5-heptamethyltrisiloxane,

corresponding to the oils of formula (I) for which R is respectively a butyl group, a propyl group or an ethyl group.

Use may also be made of volatile fluorinated solvents, such as nonafluoromethoxybutane or perfluoromethylcyclopentane.

The first and/or second composition can also comprise at least one nonvolatile oil, chosen in particular from nonvolatile hydrocarbon oils and/or silicone oils and/or fluorinated oils.

Mention may in particular be made, as nonvolatile hydrocarbon oil, of:
hydrocarbon oils of vegetable origin, such as triesters of fatty acids and of glycerol, the fatty acids of which can have varied chain lengths from C₄ to C₂₄, it being possible for these chains to be linear or branched and saturated or unsaturated; these oils are in particular wheat germ, sunflower, grape seed, sesame, maize, apricot kernel, castor, shea, avocado, olive, soybean, sweet almond, palm, rapeseed, cottonseed, hazelnut, macadamia, jojoba, alfalfa, poppy, pumpkinseed, cucumber, blackcurrant seed, evening primrose, millet, barley, quinoa, rye, safflower, candlenut, passionflower or musk rose oil; or triglycerides of caprylic/capric acids, such as those sold by Stearineries Dubois or those sold under the names Miglyol 810, 812 and 818 by Dynamit Nobel,
synthetic ethers having from 10 to 40 carbon atoms;
linear or branched hydrocarbons of mineral or synthetic origin, such as liquid petrolatum, polydecenes, hydrogenated polyisobutene, such as Parleam oil, squalane, squalene and their mixtures;
synthetic esters, such as the oils of formula R₁COOR₂ in which R₁ represents the residue of a linear or branched fatty acid comprising from 1 to 40 carbon atoms and R₂ represents a hydrocarbon chain, in particular a branched hydrocarbon chain, comprising from 1 to 40 carbon atoms, provided that R₁+R₂ is≧10, such as, for example, Purcellin oil (cetearyl octanoate), isopropyl myristate, isopropyl palmitate, C₁₂ to C₁₅ alkyl benzoate, hexyl laurate, diisopropyl adipate, isononyl isononanoate, 2-ethyihexyl palmitate, isostearyl isostearate, octanoates, decanoates or ricinoleates of alcohols or of polyalcohols, such as propylene glycol dioctanoate; hydroxylated esters, such as isostearyl lactate or diisostearyl malate; and pentaerythritol esters;
fatty alcohols comprising a branched and/or unsaturated carbon chain having from 12 to 26 carbon atoms which are liquid at ambient temperature, such as octyldodecanol, isostearyl alcohol, oleyl alcohol, 2-hexyldecanol, 2-butyloctanol or 2-undecylpentadecanol;
higher fatty acids, such as oleic acid, linoleic acid or linolenic acid;
carbonates;
acetals;
citrates;
and their mixtures.

The nonvolatile silicone oils which can be used in the composition according to the invention can be polydimethylsiloxanes (PDMSs) which are nonvolatile, polydimethylsiloxanes comprising pendent alkyl or alkoxy groups and/or alkyl or alkoxy groups at the end of the silicone chain, groups each having from 2 to 24 carbon atoms, phenylated silicones, such as phenyl trimethicones, phenyl dimethicones, phenyl(trimethylsiloxy)diphenylsiloxanes, diphenyl dimethicones, diphenyl(methyldiphenyl)trisiloxanes or (2-phenyl-ethyl)trimethyl-siloxysilicates.

The fluorinated oils which can be used in the invention are in particular fluorosilicone oils, fluorinated polyethers or fluorinated silicones, such as disclosed in the document EP-A-847 752.

According to one embodiment, the fatty phase advantageously comprises an ester oil. This ester oil can be chosen from the esters of monocarboxylic acids with monoalcohols and polyalcohols.

Advantageously, the said ester corresponds to the following formula (I):

R₁—CO—O—R₂ (I)
where R₁ represents a linear or branched alkyl radical of 1 to 40 carbon atoms, preferably of 7 to 19 carbon atoms, optionally comprising one or more ethylenic double bonds and optionally substituted,
R₂ represents a linear or branched alkyl radical of 1 to 40 carbon atoms, preferably of 3 to 30 carbon atoms and better still of 3 to 20 carbon atoms, optionally comprising one or more ethylenic double bonds and optionally substituted.

The term “optionally substituted” is understood to mean that R₁ and/or R₂ can carry one or more substituents chosen, for example, from groups comprising one or more heteroatoms chosen from O, N and S, such as amino, amine, alkoxy or hydroxyl.

Preferably, the total number of carbon atoms of R₁+R₂ is ≧9.

R₁ can represent the residue of a linear or, preferably, branched fatty acid, preferably a higher fatty acid, comprising from 1 to 40 and better still from 7 to 19 carbon atoms and R₂ can represent a linear or, preferably, branched hydrocarbon chain comprising from 1 to 40, preferably from 3 to 30 and better still from 3 to 20 carbon atoms. Again, preferably, the number of carbon atoms of R₁+R₂≧9.

Examples of the R₁ groups are those derived from the fatty acids chosen from the group consisting of acetic, propionic, butyric, caproic, caprylic, pelargonic, capric, undecanoic, lauric, myristic, palmitic, stearic, isostearic, arachidic, behenic, oleic, linolenic, linoleic, eleostearic, arachidonic and erucic acids and of their mixtures.

Examples of esters are, for example, Purcellin oil (cetearyl octanoate), isononyl isononanoate, isopropyl myristate, 2-ethylhexyl palmitate, 2-octyldodecyl stearate, 2-octyldodecyl erucate, isostearyl isostearate and the heptanoates, octanoates, decanoates or ricinoleates of alcohols or of polyalcohols, for example of fatty alcohols.

Advantageously, the esters are chosen from the compounds of the above formula (I) in which R₁ represents an unsubstituted linear or branched alkyl group of 1 to 40 carbon atoms, preferably of 7 to 19 carbon atoms, optionally comprising one or more ethylenic double bonds and R₂ represents an unsubstituted linear or branched alkyl group of 1 to 40 carbon atoms, preferably of 3 to 30 carbon atoms and better still of 3 to 20 carbon atoms, optionally comprising one or more ethylenic double bonds.

Preferably, R₁ is an unsubstituted branched alkyl group of 4 to 14 carbon atoms, preferably of 8 to 10 carbon atoms, and R₂ is an unsubstituted branched alkyl group of 5 to 15 carbon atoms, preferably of 9 to 11 carbon atoms. Preferably, in the formula (I), R₁—CO— and R₂ have the same number of carbon atoms and derive from the same radical, preferably unsubstituted branched alkyl, for example isononyl, that is to say that, advantageously, the molecule of ester oil is symmetrical.

The ester oil will preferably be chosen from the following compounds:
isononyl isononanoate,
cetearyl octanoate,
isopropyl myristate,
2-ethylhexyl palmitate,
2-octyldodecyl stearate,
2-octyldodecyl erucate,
isostearyl isostearate.

In the case where the compositions are intended to be applied to the lips, use may in particular be made of a “viscous” oil, that is to say an oil having a viscosity at 25° C. advantageously of greater than or equal to 200 cSt, in particular of greater than or equal to 500 cSt, indeed even of greater than or equal to 1000 cSt. The viscous oil advantageously exhibits a molecular weight of greater than or equal to 600 g/mol, for example of greater than or equal to 700, indeed even 800, indeed even 900 g/mol.

The dynamic viscosity at 25° C. of the viscous oil can be measured with a Mettler RM 180 rotational viscometer, the density of the oil being taken into consideration in carrying out the conversion to cSt.

The Mettler RM 180 device (Rheomat) can be equipped with various spindles according to the order of magnitude of the viscosity which it is desired to measure. For a viscosity of between 0.18 and 4.02 Pa·s, the device is equipped with a spindle 3. For a viscosity of between 1 and 24 Pa·s, the device is equipped with a spindle 4 and, for a viscosity of between 8 and 122 Pa·s, the device is equipped with a spindle 5. The viscosity is read on the device in deviation units (DU). Reference is subsequently made to grafts supplied with the measurement device in order to obtain the corresponding value in poises and then to carry out the conversion to stokes.

The rotational speed of the spindle is 200 revolutions/min.

From the moment when the spindle is set rotating, at a constant imposed rotational speed (in the case in point, 200 revolutions/min), the viscosity value of the oil can vary over time. Measurements are taken at regular time intervals until they become constant. The value of the viscosity which has become constant over time is the value selected as being the value of the dynamic viscosity of the viscous oil.

This oil can be chosen from:

a) silicone oils, such as
polydimethylsiloxanes (PDMSs), optionally comprising a C3-C₄₀ alkyl or C₃-C₄₀ alkoxy chain or a phenyl radical; polydimethylsiloxanes comprising phenylated radicals can be chosen from phenyl trimethicones;
optionally fluorinated polyalkylmethylsiloxanes, such as polymethyltrifluoropropyldimethylsiloxanes,
polyalkylmethylsiloxanes substituted by functional groups, such as hydroxyl, thiol and/or amine groups;
polysiloxanes modified by fatty acids, fatty alcohols or polyoxyalkylenes,
their mixtures,

b) nonpolar hydrocarbon oils, such as squalene, linear or branched hydrocarbons, such as paraffin, petrolatum and naphthalene oils, hydrogenated or partially hydrogenated polyisobutene, isoeicosane, squalane, decene/butene copolymers, polybutene/polyisobutene copolymers, in particular Indopol L-14, polydecenes, such as Puresyn 10, and their mixtures.

The fatty phase can represent from 5 to 80% by weight, with respect to the total weight of the composition, preferably from 10 to 60% and more preferably still from 15 to 50% by weight.

According to one embodiment, the first composition and/or the second composition employed in the process according to the invention are anhydrous, that is to say devoid of water other than the residual water contributed by some compounds.

Aqueous Phase

The first and/or the second composition can comprise an aqueous phase.

The aqueous phase can be composed essentially of water; it can also comprise a mixture of water and of water-miscible solvent (miscibility in water of greater the 50% by weight at 25° C.), such as lower monoalcohols having from 1 to 5 carbon atoms, for example ethanol or isopropanol, glycols having from 2 to 8 carbon atoms, such as propylene glycol, ethylene glycol, 1,3-butylene glycol or dipropylene glycol, C₃-C₄ ketones, C₂-C₄ aldehydes and their mixtures.

The aqueous phase can, in this case, represent from 5 to 95% by weight, with respect to the total weight of the composition comprising it, preferably from 10 to 85% by weight.

Solid or Pasty Fatty Substances

The composition according to the invention can also comprise at least one fatty substance which is solid at ambient temperature chosen in particular from waxes, pasty fatty substances and their mixtures. These fatty substances can be of animal, vegetable, mineral or synthetic origin.

Wax

The composition according to the invention can comprise a wax or a mixture of waxes.

The wax under consideration in the context of the present invention is generally a lipophilic compound which is solid at ambient temperature (25° C.), which exhibits a reversible solid/liquid change in state and which has a melting point of greater than or equal to 30° C. which can range up to 120° C.

On bringing the wax to the liquid state (melting), it is possible to render it miscible with oils and to form a microscopically homogeneous mixture but, on bringing the temperature of the mixture back to ambient temperature, recrystallization of the wax in the oils of the mixture is obtained.

In particular, the waxes suitable for the invention can exhibit a melting point of greater than approximately 45° C. and in particular of greater than 55° C.

The melting point of the wax can be measured using a differential scanning calorimeter (DSC), for example the calorimeter sold under the name DSC 30 by Mettler.

The measurement protocol is as followed:

A 15 mg sample of the product placed in a crucible is subjected to a first rise in temperature ranging from 0° C. to 120° C. at a heating rate of 10° C./ minute, is then cooled from 120° C. to 0° C. at a cooling rate of 10° C./minute and, finally, is subjected to a second rise in temperature ranging from 0° C. to 120° C. at a heating rate of 5° C./minute. During the second rise in temperature, the variation in the difference in power absorbed by the empty crucible and by the crucible comprising the sample of product is measured as a function of the temperature. The melting point of the compound is the value of the temperature corresponding to the tip of the peak of the curve representing the variation in the difference in powder absorbed as a function of the temperature.

The waxes capable of being used in the compositions according to the invention are chosen from waxes of animal, vegetable, mineral or synthetic origin, and their mixtures, which are deformable or nondeformable solids at ambient temperature.

The wax can also exhibit a hardness ranging from 0.05 MPa to 30 MPa and preferably ranging from 6 MPa to 15 MPa. The hardness is determined by the measurement of the compressive force measured at 20° C. using a texture analyser sold under the name TA-TX2i by Rheo, equipped with a stainless steel cylinder with a diameter of 2 mm which is displaced at the measuring rate of 0.1 mm/s, and which penetrates the wax to a penetration depth of 0.3 mm.

The measurement protocol is as follows:

The wax is melted to a temperature equal to the melting point of the wax +20° C. The molten wax is cast in a receptacle with a diameter of 30 mm and a depth of 20 mm. The wax is recrystallized at ambient temperature (25° C.) for 24 hours and then the wax is stored at 20° C. for at least 1 hour before measuring the hardness. The value of the hardness is the maximum compressive force measured divided by the surface area of the cylinder of the texture analyser in contact with the wax.

Use may in particular be made of hydrocarbon waxes, such as beeswax, lanolin wax and Chinese insect waxes; rice wax, carnauba wax, candelilla wax, ouricury wax, esparto wax, cork fibre wax, sugarcane wax, Japan wax and sumac wax; montan wax, microcrystalline waxes, paraffin waxes and ozokerite; polyethylene waxes, waxes obtained by the Fischer-Tropsch synthesis and waxy copolymers, and also their esters.

Mention may also be made of the waxes obtained by catalytic hydrogenation of animal or vegetable oils having linear or branched C₈-C₃₂ fatty chains.

Mention may in particular be made, among these, of hydrogenated jojoba oil, isomerized jojoba oil, such as the trans-isomerized partially hydrogenated jojoba oil manufactured or sold by Desert Whale under the commercial reference Iso-Jojoba-50®, hydrogenated sunflower oil, hydrogenated castor oil, hydrogenated coconut oil, hydrogenated lanolin oil, di(1,1,1-trimethylolpropane)tetrastearate, sold under the name “Hest 2T-4S” by Heterene, or di(1,1,1-trimethylolpropane)tetrabehenate, sold under the name “Hest 2T-4B” by Heterene.

Mention may also be made of silicone waxes, such as alkyl or alkoxy dimethicones having from 16 to 45 carbon atoms, or fluorinated waxes.

Use may also be made of the wax obtained by hydrogenation of olive oil esterified with stearyl alcohol sold under the name “Phytowax Olive 18 L 57” or else of the waxes obtained by hydrogenation of castor oil esterified with cetyl alcohol sold under the names “Phytowax castor 16L64” and “Phytowax castor 22L73” by Sophim. Such waxes are described in Application FR-A-2 792 190.

According to a specific embodiment, the compositions according to the invention can comprise at least one wax known as “tacky wax”, that is to say having a tack of greater than or equal to 0.7 N.s and a hardness of less than or equal to 3.5 MPa.

The use of a tacky wax can in particular make it possible to obtain a cosmetic composition which is easily applied to keratinous fibres, which has good attachment to keratinous fibres and which results in the formation of a smooth, homogeneous and thickening makeup.

The tacky wax used can in particular have a tack ranging from 0.7 N.s to 30 N.s, in particular of greater than or equal to 1 N.s, in particular ranging from 1 N.s to 20 N.s, especially of greater than or equal to 2 N.s, in particular ranging from 2 N.s to 10 N.s, and especially ranging from 2 N.s to 5 N.s.

The tack of the wax is determined by the measurement of the change in the force (compressive force or stretching force) as a function of the time at 20° C. using the texture analyser sold under the name “TA-TX2i®” by Rheo, equipped with a spindle made of acrylic polymer in the shape of a cone forming an angle of 45°.

The measurement protocol is as follows:

The wax is melted at a temperature equal to the melting point of the wax +10° C. The molten wax is cast in a receptacle with a diameter of 25 mm and a depth of 20 mm. The wax is recrystallized at ambient temperature (25° C.) for 24 hours, so that the surface of the wax is flat and smooth, and then the wax is stored at 20° C. for at least 1 hour before measuring the tack.

The spindle of the texture analyser is displaced at the rate of 0.5 mm/s and then penetrates the wax to a penetration depth of 2 mm. When the spindle has penetrated the wax to a depth of 2 mm, the spindle is held stationary for 1 second (corresponding to the relaxation time) and is then withdrawn at the rate of 0.5 mm/s.

During the relaxation time; the force (compressive force) strongly decreases until it becomes zero and then, during the withdrawal of the spindle, the force (stretching force) becomes negative to subsequently again increase towards the value of 0. The tack corresponds to the integral of the curve of the force as a function of the time for the part of the curve corresponding to the negative values of the force (stretching force). The value of the tack is expressed in N.s.

The tacky wax which can be used generally has a hardness of less than or equal to 3.5 MPa, in particular ranging from 0.01 MPa to 3.5 MPa, especially ranging from 0.05 MPa to 3 MPa, indeed even also ranging from 0.1 MPa to 2.5 MPa.

The hardness is measured according to the protocol described above.

Use may be made, as tacky wax, of a C₂₀-C₄₀ alkyl (hydroxystearyloxy)stearate (the alkyl group comprising from 20 to 40 carbon atoms), alone or as a mixture, in particular a C₂₀-C40 alkyl 12-(12′-hydroxystearyloxy)stearate.

Such a wax is sold in particular under the names “Kester Wax K 82 P®” and “Kester Wax K 80 P®” by Koster Keunen.

The abovementioned waxes generally exhibit a starting melting point of less than 45° C.

The wax or waxes can be present in the form of an aqueous wax microdispersion. The term “aqueous wax microdispersion” is understood to mean an aqueous dispersion of wax particles in which the size of the said wax particles is less than or equal to approximately 1 μm.

Wax microdispersions are stable dispersions of colloidal wax particles and are described in particular in “Microemulsions Theory and Practice”, edited by L. M. Prince, Academic Press (1977), pages 21-32.

In particular, these wax microdispersions can be obtained by melting the wax in the presence of a surfactant and optionally of a portion of the water and then gradually adding hot water with stirring. The intermediate formation of an emulsion of the water-in-oil type, followed by phase inversion, with a microemulsion of oil-in-water type finally being obtained, is observed. On cooling, a stable microdispersion of solid colloidal wax particles is obtained.

The wax microdispersions can also be obtained by stirring the mixture of wax, of surfactant and of water using stirring means, such as ultrasound, a high pressure homogenizer or turbine mixers.

The particles of the wax microdispersion preferably have mean sizes of less than 1 μm (in particular ranging from 0.02 μm to 0.99 μm), preferably of less than 0.5 μm (in particular ranging from 0.06 μm to 0.5 μm).

These particles are composed essentially of a wax or of a mixture of waxes. However, they can comprise a minor proportion of oily and/or pasty fatty additives, a surfactant and/or a conventional fat-soluble additive/active principle.

The term “pasty fatty substances” is understood to mean a lipophilic fatty compound comprising, at a temperature of 23° C., a liquid fraction and a solid fraction.

The said pasty compound preferably has a hardness at 20° C. ranging from 0.001 to 0.5 MPa, preferably from 0.002 to 0.4 MPa.

The hardness is measured according to a method of penetration of a probe into a sample of compound and in particular using a texture analyser (for example, the TA-XT2i from Rheo) equipped with a stainless steel cylinder with a diameter of 2 mm. The hardness measurement is carried out at 20° C. at the centre of 5 samples. The cylinder is introduced into each sample at a pre-rate of 1 mm/s and then at a measuring rate of 0.1 mm/s, the depth of penetration being 0.3 mm. The value recorded for the hardness is that of the maximum peak.

The liquid fraction of the pasty compound measured at 23° C. preferably represents 9 to 97% by weight of the compound. This liquid fraction at 23° C. preferably represents between 15 and 85%, more preferably between 40 and 85%, by weight. The liquid fraction by weight of the pasty compound at 23° C. is equal to the ratio of the enthalpy of fusion consumed at 23° C. to the enthalpy of fusion of the pasty compound.

The enthalpy of fusion of the pasty compound is the enthalpy consumed by the compound to change from the solid state to the liquid state. The pasty compound is “in the solid state” when the whole of its mass is in the crystalline solid form. The pasty compound is “in the liquid state” when the whole of its mass is in the liquid form.

The enthalpy of fusion of the pasty compound is equal to the area under the curve of the thermogram obtained using a differential scanning calorimeter (DSC), such as the calorimeter sold under the name MDSC 2920 by TA Instrument, with a rise in temperature of 5 or 10° C. per minute, according to Standard ISO 11357-3:1999. The enthalpy of fusion of the pasty compound is the amount of energy necessary to change the compound from the solid state to the liquid state. It is expressed in J/g.

The enthalpy of fusion consumed at 23° C. is the amount of energy absorbed by the sample to change from the solid state to the state which it exhibits at 23° C., composed of a liquid fraction and of a solid fraction.

The liquid fraction of the pasty compound measured at 32° C. preferably represents from 30 to 100% by weight of the compound, preferably from 80 to 100%, more preferably from 90 to 100% by weight of the compound. When the liquid fraction of the pasty compound measured at 32° C. is equal to 100%, the temperature of the end of the melting range of the pasty compound is less than or equal to 32° C.

The liquid fraction of the pasty compound measured at 32° C. is equal to the ratio of the enthalpy of fusion consumed at 32° C. to the enthalpy of fusion of the pasty compound. The enthalpy of fusion consumed at 32° C. is calculated in the same way as the enthalpy of fusion consumed at 23° C.

The pasty substances are generally hydrocarbon compounds, such as lanolins and their derivatives, or also PDMSs.

The nature and the amount of the solid substances depend on the mechanical properties and textures desired. By way of indication, each first or second composition can comprise from 0.1 to 70% by weight of waxes, with respect to the total weight of the composition, better still from 1 to 60% by weight and even better still from 5 to 40% by weight.

Film-Forming Polymer

The first and/or second composition can comprise a film-forming polymer. According to the present invention, the term “film-forming polymer” is understood to mean a polymer capable of forming, alone or in the presence of an additional agent which is able to form a film, a continuous film which adheres to a support, in particular to keratinous substances.

The film-forming polymer can be present in each composition according to the invention in a content of dry matter (or active materials) ranging from 0.1% to 30% by weight, with respect to the total weight of the composition, preferably from 0.5% to 20% by weight and better still from 1% to 15% by weight.

Mention may be made, among the film-forming polymers which can be used in the composition of the present invention, of synthetic polymers of radical type or of polycondensate type, polymers of natural origin, and their mixtures.

The term “radical film-forming polymer” is understood to mean a polymer obtained by polymerization of monomers possessing unsaturation, in particular ethylenic unsaturation, each monomer being capable of homopolymerizing (unlike polycondensates).

The film-forming polymers of radical type can in particular be vinyl polymers or copolymers, in particular acrylic polymers.

The film-forming vinyl polymers can result from the polymerization of monomers possessing ethylenic unsaturation having at least one acid group and/or of the esters of these acidic monomers and/or of the amides of these acidic monomers.

Use may be made, as monomer carrying an acid group, of unsaturated α,β-ethylenic carboxylic acids, such as acrylic acid, methacrylic acid, crotonic acid, maleic acid or itaconic acid. Use is preferably made of (meth)acrylic acid and crotonic acid and more preferentially of (meth)acrylic acid.

The esters of acidic monomers are advantageously chosen from esters of (meth)acrylic acid (also known as (meth)acrylates), in particular alkyl (meth)acrylates, especially C₁-C₃₀ alkyl (meth)acrylates, preferably C₁-C₂₀ alkyl (meth)acrylates, aryl (meth)acrylates, in particular C₆-C₁₀ aryl (meth)acrylates, hydroxyalkyl (meth)acrylates, in particular C₂-C₆ hydroxyalkyl (meth)acrylates.

Mention may be made, among alkyl (meth)acrylates, of methyl methacrylate, ethyl methacrylate, butyl methacrylate, isobutyl methacrylate, 2-ethylhexyl methacrylate, lauryl methacrylate or cyclohexyl methacrylate.

Mention may be made, among hydroxyalkyl (meth)acrylates, of hydroxyethyl acrylate, 2-hydroxypropyl acrylate, hydroxyethyl methacrylate or 2-hydroxypropyl methacrylate.

Mention may be made, among aryl (meth)acrylates, of benzyl acrylate and phenyl acrylate.

Esters of (meth)acrylic acid which are particularly preferred are alkyl (meth)acrylates.

According to the present invention, the alkyl group of the esters can be either fluorinated or perfluorinated, that is to say that a portion or all of the hydrogen atoms of the alkyl group are substituted by fluorine atoms.

Mention may be made, as amides of the acidic monomers, for example, of (meth)acrylamides, in particular N-alkyl(meth)acrylamides, especially N-(C₂-C₁₂ alkyl)(meth)acrylamides. Mention may be made, among N-alkyl(meth)acrylamides, of N-ethylacrylamide, N-(t-butyl)acrylamide, N-(t-octyl)acrylamide and N-undecylacrylamide.

The film-forming vinyl polymers can also result from the homopolymerization or from the copolymerization of monomers chosen from vinyl esters and styrene monomers. In particular, these monomers can be polymerized with acidic monomers and/or their esters and/or their amides, such as those mentioned above.

Mention may be made, as examples of vinyl esters, of vinyl acetate, vinyl neodecanoate, vinyl pivalate, vinyl benzoate and vinyl t-butylbenzoate.

Mention may be made, as styrene monomers, of styrene and α-methylstyrene.

Mention may be made, among film-forming polycondensates, of polyurethanes, polyesters, polyesteramides, polyamides, epoxy ester resins or polyureas.

The polyurethanes can be chosen from anionic, cationic, nonionic or amphoteric polyurethanes, polyurethane-acrylics, polyurethane-polyvinylpyrrolidones, polyester-polyurethanes, polyether-polyurethanes, poly-ureas, polyurea-polyurethanes, a their blends.

The polyesters can be obtained in a known way by polycondensation of dicarboxylic acids with polyols, in particular diols.

The dicarboxylic acid can be aliphatic, alicyclic or aromatic. Mention may be made, as examples of such acids, of oxalic acid, malonic acid, dimethylmalonic acid, succinic acid, glutaric acid, adipic acid, pimelic acid, 2,2-dimethylglutaric acid, azelaic acid, suberic acid, sebacic acid, fumaric acid, maleic acid, itaconic acid, phthalic acid, dodecanedioic acid, 1,3-cyclohexanedicarboxylic acid, 1,4-cyclohexane-dicarboxylic acid, isophthalic acid, terephthalic acid, 2,5-norbornanedicarboxylic acid, diglxcolic acid, thiodipropionic acid, 2,5-naphthalenedicarboxylic acid or 2,6-naphthalenedicarboxylic acid. These dicarboxylic acid monomers can be used alone or as a combination of at least two dicarboxylic acid monomers. The choice is preferentially made, among these monomers, of phthalic acid, isophthalic acid or terephthalic acid.

The diol can be chosen from aliphatic, alicyclic or aromatic diols. Use is preferably made of a diol chosen from ethylene glycol, diethylene glycol, triethylene glycol, 1,3-propanediol, cyclohexanedimethanol or 1,4-butanediol. Use may be made, as other polyols, of glycerol, pentaerythritol, sorbitol or trimethylol-propane.

The polyesteramides can be obtained in an analogous way to the polyesters, by polycondensation of diacids with diamines or aminoalcohols. Use may be made, as diamine, of ethylenediamine, hexamethylenediamine, meta-phenylenediamine or para-phenylenediamine. Use may be made, as aminoalcohol, of monoethanolamine.

The polyester can additionally comprise at least one monomer carrying at least one —SO₃M group, with M representing a hydrogen atom, an NH₄⁺ ammonium ion or a metal ion, such as, for example, an Na⁺, Li⁺, K⁺, Mg²⁺, Ca²⁺, Cu²⁺, Fe²⁺ or Fe³⁺ ion. Use may in particular be made of a bifunctional aromatic monomer comprising such an —SO₃M group.

The aromatic nucleus of the bifunctional aromatic monomer additionally carrying an —SO₃M group as described above can be chosen, for example, from the benzene, naphthalene, anthracene, diphenyl, oxydiphenyl, sulphonyldiphenyl or methylenediphenyl nuclei. Mention may be made, as example of bifunctional aromatic monomer additionally carrying an —SO₃M group, of sulphoisophthalic acid, sulphoterephthalic acid, sulphophthalic acid or 4-sulphonaphthalene-2,7-dicarboxylic acid.

Preference is given to the use of copolymers based on isophthalate/sulphoisophthalate and more particularly to copolymers obtained by condensation of diethylene glycol, cyclohexanedimethanol, isophthalic acid and sulphoisophthalic acid.

The optionally modified polymers of natural origin can be chosen from shellac resin, gum sandarac, dammars, elemis, copals, cellulose polymers and their blends.

According to a first embodiment of the composition according to the invention, the film-forming polymer can be a water-soluble polymer and can be present in an aqueous phase of the composition; the polymer is thus dissolved in the aqueous phase of the composition.

According to another alternative embodiment of the composition according to the invention, the film-forming polymer can be a polymer dissolved in a liquid fatty phase comprising oils or organic solvents, such as those described above (the film-forming polymer is then described as a fat-soluble polymer). Preferably, the liquid fatty phase comprises a volatile oil, optionally as a mixture with a non-volatile oil, it being possible for the oils to be chosen from the oils mentioned above.

Mention may be made, as examples of fat-soluble polymer, of copolymers of vinyl ester (the vinyl group being directly connected to the oxygen atom of the ester group and the vinyl ester having a saturated, linear or branched, hydrocarbon radical of 1 to 19 carbon atoms bonded to the carbonyl of the ester group) and of at least one other monomer which can be a vinyl ester (other than the vinyl ester already present), an α-olefin (having from 8 to 28 carbon atoms), an alkyl vinyl ether (the alkyl group of which comprises from 2 to 18 carbon atoms) or an allyl or methallyl ester (having a saturated, linear or branched, hydrocarbon radical of 1 to 19 carbon atoms bonded to the carbonyl of the ester group).

These copolymers can be crosslinked using crosslinking agents which can be either of the vinyl type or of the allyl or methallyl type, such as tetraallyloxyethane, divinylbenzene, divinyl octanedioate, divinyl dodecane-dioate and divinyl octadecanedioate.

Mention may be made, as examples of these copolymers, of the following copolymers: vinyl acetate/allyl stearate, vinyl acetate/vinyl laurate, vinyl acetate/vinyl stearate, vinyl acetate/octadecene, vinyl acetate/octadecyl vinyl ether, vinyl propionate/allyl laurate, vinyl propionate/vinyl laurate, vinyl stearate/1-octadecene, vinyl acetate/1-dodecene, vinyl stearate/ethyl vinyl ether, vinyl propionate/cetyl vinyl ether, vinyl stearate/allyl acetate, vinyl 2,2-dimethyloctanoate/vinyl laurate, allyl 2,2-dimethylpentanoate/vinyl laurate, vinyl dimethyl-propionate/vinyl stearate, allyl dimethylpropionate/vinyl stearate, vinyl propionate/vinyl stearate, crosslinked with 0.2% of divinylbenzene, vinyl dimethylpropionate/vinyl laurate, crosslinked with 0.2% of divinylbenzene, vinyl acetate/octadecyl vinyl ether, crosslinked with 0.2% of tetraallyloxyethane, vinyl acetate/allyl stearate, crosslinked with 0.2% of divinylbenzene, vinyl acetate/1-octadecene, crosslinked with 0.2% of divinylbenzene, and allyl propionate/allyl stearate, crosslinked with 0.2% of divinylbenzene.

Mention may also be made, as fat-soluble film-forming polymers, of fat-soluble copolymers and in particular those resulting from the copolymerization of vinyl esters having from 9 to 22 carbon atoms or of alkyl acrylates or methacrylates, the alkyl radicals having from 10 to 20 carbon atoms.

Such fat-soluble copolymers can be chosen from copolymers of poly(vinyl stearate), of poly(vinyl stearate) crosslinked using divinylbenzene, diallyl ether or diallyl phthalate, copolymers of poly(stearyl(meth)acrylate), of poly(vinyl laurate), of poly(lauryl(meth)acrylate), it being possible for these poly(meth)acrylates to be crosslinked using ethylene glycol dimethacrylate or tetraethylene glycol dimethacrylate.

The fat-soluble copolymers defined above are known and are disclosed in particular in Application FR-A-2 232 303; they can have a weight-average molecular weight ranging from 2000 to 500 000 and preferably from 4000 to 200 000.

Mention may also be made of fat-soluble homopolymers and in particular of those resulting from the homopolymerization of vinyl esters having from 9 to 22 carbon atoms or of alkyl acrylates or methacrylates, the alkyl radicals having from 2 to 24 carbon atoms.

Mention may in particular be made, as examples of fat-soluble homopolymers, of: poly(vinyl laurate) and poly(lauryl(meth)acrylate)s, it being possible for these poly(meth)acrylates to be crosslinked using ethylene glycol or tetraethylene glycol dimethacrylate.

According to an advantageous embodiment, the first composition of the process according to the invention comprises at least one poly(vinyl laurate) film-forming polymer.

Mention may also be made, as fat-soluble film-forming polymers which can be used in the invention, of polyalkylenes and in particular copolymers of C₂-C₂₀ alkenes, such as polybutene, alkylcelluloses with a saturated or unsaturated and linear or branched C₁ to C₈ alkyl radical, such as ethylcellulose and propylcellulose, copolymers of vinylpyrrolidone (VP) and in particular copolymers of vinylpyrrolidone and of C₂ to C₄₀ alkene and better still C₃ to C₂₀ alkene. Mention may be made, as examples of VP copolymer which can be used in the invention, of the VP/vinyl acetate, VP/ethyl methacrylate, VP/ethyl methacrylate/methacrylic acid, VP/eicosene, VP/hexadecene, VP/triacontene, VP/styrene or VP/acrylic acid/lauryl methacrylate copolymer or butylated polyvinylpyrrolidone (PVP).

Mention may also be made of silicone resins, generally soluble or swellable in silicone oils, which are crosslinked polyorganosiloxane polymers. The nomenclature of silicone resins is known under the name of “MDTQ”, the resin being described according to the various siloxane monomer units which it comprises, each of the letters “MDTQ” characterizing one type of unit.

Mention may be made, as examples of commercially available polymethylsilsesquioxane resins, of those which are sold:
by Wacker under the reference Resin MK, such as Belsil PMS MK;
by Shin-Etsu under the references KR-220L.

Mention may be made, as siloxysilicate resins, of trimethylsiloxysilicate (TMS) resins, such as those sold under the reference SR1000 by General Electric or under the reference TMS 803 by Wacker. Mention may also be made of trimethylsiloxysilicate resins sold in a solvent, such as cyclomethicone, sold under the names “KF-7312J” by Shin-Etsu or “DC 749” or “DC 593” by Dow Corning.

Mention may also be made of copolymers of silicone resins, such as those mentioned above, with polydimethylsiloxanes, such as the pressure-sensitive adhesive copolymers sold by Dow Corning under the reference BIO-PSA and disclosed in the document U.S. Pat. No. 5,162,410 or the silicone copolymers resulting from the reaction of a silicone resin, such as those described above, and of a diorganosiloxane, such as are disclosed in the document WO 2004/073626.

Use may also be made of silicone polyamides of the polyorganosiloxane type, such as those described in the documents U.S. Pat. No. 5,874,069, U.S. Pat. No. 5,919,441, U.S. Pat. No. 6,051,216 and U.S. Pat. No. 5,981,680.

These silicone polymers can belong to the following two families:
polyorganosiloxanes comprising at least two groups capable of establishing hydrogen interactions, these two groups being situated in the chain of the polymer, and/or
polyorganosiloxanes comprising at least two groups capable of establishing hydrogen interactions, these two groups being situated on grafts or branchings.

According to one embodiment of the invention, the film-forming polymer is a film-forming linear block ethylenic polymer which preferably comprises at least one first block and at least one second block having different glass transition temperatures (Tg), the said first and second blocks being connected to one another via an intermediate block comprising at least one constituent monomer of the first block and at least one constituent monomer of the second block.

Advantageously, the first and second blocks of the block polymer are incompatible with one another.

Such polymers are disclosed, for example, in the documents EP 1 411 069 or WO04/028488.

The film-forming polymer can also be present in the composition in the form of particles in dispersion in an aqueous phase or in a nonaqueous solvent phase, generally known under the name of latex or pseudolatex. The techniques for the preparation of these dispersions are well known to a person skilled in the art.

Use may be made, as aqueous film-forming polymer dispersion, of acrylic dispersions, sold under the names Neocryl XK-90®, Neocryl A-1070®, Neocryl A-1090®, Neocryl BT-62®, Neocryl A-1079® and Neocryl A-523® by Avencia Neoresins, Dow Latex 432® by Dow Chemical, Daitosol 5000 AD® or Daitosol 5000 SJ® by Daito Kasey Kogyo; Syntran 5760® by Interpolymer, Allianz OPT by Röhm & Haas, aqueous dispersions of acrylic or styrene/acrylic polymers, sold under the trade name Joncryl® by Johnson Polymer, or aqueous dispersions of polyurethane, sold under the names Neorez R-981® and Neorez R-974® by Avecia-Neoresins, Avalure UR-405®, Avalure UR-410®, Avalure UR-425®, Avalure UR-450®, Sancure 875®, Sancure 861®, Sancure 878® and Sancure 2060® by Goodrich, Impranil 85® by Bayer, Aquamere H-1511® by Hydromer; sulphopolyesters, sold under the trade name Eastman AQ® by Eastman Chemical Products, vinyl dispersions, such as Mexomer PAM® from Chimex, and their blends.

Mention may be made, as examples of nonaqueous dispersions of film-forming polymer, of acrylic dispersions in isododecane, such as Mexomer PAP® from Chimex, dispersions of particles of a grafted ethylenic polymer, preferably an acrylic polymer, in a liquid fatty phase, the ethylenic polymer advantageously being dispersed in the absence of additional stabilizer at the surface of the particles, such as disclosed in particular in the document WO 04/055081.

The composition according to the invention can comprise a plasticizing agent favourable to the formation of a film with the film-forming polymer. Such a plasticizing agent can be chosen from any compound known to a person skilled in the art as being capable of fulfilling the desired role.

Colouring Materials

The first and second compositions employed in the method according to the invention can comprise at least one colouring material chosen, for example, from pigments, pearlescent agents, dyes, effect materials and their mixtures.

These colouring materials can be present in a content ranging from 0.01% to 50% by weight, preferably from 0.01% to 30% by weight, with respect to the weight of each first and second composition.

The pigments of use in the present invention can be provided in the form of a pigment paste or powder.

The term “dyes” should be understood as meaning compounds, generally organic compounds, which are soluble in at least one oil or in one aqueous/alcoholic phase.

The term “pigments” should be understood as meaning white or coloured and inorganic or organic particles which are insoluble in the aqueous or medium and which are intended to colour and/or opacify the resulting film.

The term “pearlescent agents” or “pearlescent pigments” should be understood as meaning coloured particles of any shape, iridescent or noniridescent, produced in particular by certain shellfish in their shells or synthesized and which exhibit an effect of colour optical interference.

The pigments can be dispersed in their product by virtue of a dispersing agent.

The dispersing agent serves to protect the dispersed particles from the agglomeration or flocculation thereof. This dispersing agent can be a surfactant, an oligomer, a polymer or a mixture of several of them carrying one or more functionalities having a strong affinity for the surface of the particles to be dispersed. In particular, they can become attached physically or chemically to the surface of the pigments. These dispersants additionally exhibit at least one functional group compatible or soluble in the continuous medium. Use is made in particular of esters of 12-hydroxystearic acid, in particular, and of C₈ to C₂₀ fatty acid and of polyol, such as glycerol or diglycerol, for example the stearate of poly(12-hydroxystearic acid) with a molecular weight of approximately 750 g/mol, such as that sold under the name of Solsperse 21 000 by Avecia, polyglyceryl-2 dipolyhydroxystearate (CTFA name), sold under the reference Dehymyls PGPH by Henkel, or polyhydroxystearic acid, such as that sold under the reference Arlacel P100 by Uniqema, and their mixtures.

Mention may be made, as other dispersant which can be used in the composition of the invention, of the quaternary ammonium derivatives of polycondensed fatty acids, such as Solsperse 17 000, sold by Avecia, or polydimethylsiloxane/oxypropylene mixtures, such as those sold by Dow Corning under the references DC2-5185 or DC2-5225 C.

The polydihydroxystearic acid and the esters of 12-hydroxystearic acid are preferably intended for a hydrocarbon or fluorinated medium, while the oxyethylene/oxypropylenated dimethylsiloxane mixtures are preferably intended for a silicone medium.

Mention may be made, among inorganic pigments, of titanium dioxide, optionally treated at the surface, zirconium or cerium oxides, and also zinc, iron (black, yellow or red) or chromium oxides, manganese violet, ultramarine blue, chromium hydrate and ferric blue, and metal powders, such as aluminium powder or copper powder.

Mention may be made, among organic pigments, of carbon black, pigments of D & C type and lakes, based on cochineal carmine, of barium, strontium, calcium or aluminium.

Mention may also be made of effect pigments, such as particles comprising an organic or inorganic and natural or synthetic substrate, for example glass, acrylic resins, polyester, polyurethane, polyethylene terephthalate, ceramics or aluminas, the said substrate being or not being covered with metal substances, such as aluminium, gold, silver, platinum, copper or bronze, or with metal oxides, such as titanium dioxide, iron oxide or chromium oxide, and their mixtures.

The pearlescent pigments can be chosen from mica covered with titanium oxide or with bismuth oxychloride, titanium oxide-coated mica covered with iron oxides, titanium oxide-coated mica covered with in particular ferric blue or chromium oxide, titanium oxide-coated mica covered with an organic pigment of the abovementioned type, and also pearlescent pigments based on bismuth oxychloride. Use may also be made of interference pigments, in particular liquid crystal or multilayer pigments.

The compositions according to the invention can comprise at least one filler, in particular in a content ranging from 0.01% to 50% by weight, with respect to the total weight of each composition, preferably ranging from 0.01% to 30% by weight. The fillers can be inorganic or organic and of any shape, platelet, spherical or oblong, whatever the crystallographic form (for example, sheet, cubic, hexagonal, orthorhombic, and the like). Mention may be made of talc, mica, silica, kaolin, powders formed of polyamide (Nylon®) (Orgasol® from Atochem), of poly-β-alanine and of polyethylene, powders formed of tetrafluoroethylene polymers (Teflon®), lauroyllysine, starch, boron nitride, hollow polymer microspheres, such as those of poly(vinylidene chloride)/acrylonitrile, for example Expancel® (Nobel Industry) or of acrylic acid copolymers (Polytrap® from Dow Corning) and silicone resin microbeads (Tospearls® from Toshiba, for example), particles formed of polyorganosiloxane elastomers, precipitated calcium carbonate, magnesium carbonate, basic magnesium carbonate, hydroxyapatite, hollow silica microspheres (Silica Beads® from Maprecos), glass or ceramic microcapsules, or metal soaps derived from organic carboxylic acids having from 8 to 22 carbon atoms, preferably from 12 to 18 carbon atoms, for example zinc stearate, magnesium stearate, lithium stearate, zinc laurate or magnesium myristate.

The compositions according to the invention can also comprise ingredients commonly used in cosmetics, such as vitamins, thickeners, lipophilic or hydrophilic gelling agents, trace elements, softeners, sequestering agents, fragrances, basifying or acidifying agents, preservatives, sunscreens, surfactants, antioxidants, fibres, care agents or their mixtures.

The gelling agents which can be used in the compositions according to the invention can be polymeric or molecular, organic or inorganic and hydrophilic or lipophilic gelling agents.

Mention may be made, as inorganic lipophilic gelling agent, of optionally modified clays, such as hectorites modified by a C₁₀ to C₂₂ fatty acid ammonium chloride, such as hectorite modified by distearyldimethylammonium chloride, such as, for example, that sold under the name of “Bentone 38V®” by Elementis.

Mention may also be made of pyrogenic silica optionally treated hydrophobically at the surface, the size of the particles of which is less than 1 μm. This is because it is possible to chemically modify the surface of the silica by chemical reaction, resulting in a reduction in the number of silanol groups present at the surface of the silica. It is possible in particular to substitute silanol groups by hydrophobic groups: a hydrophobic silica is then obtained. The hydrophobic groups can be:
trimethylsiloxyl groups, which are obtained in particular by treatment of pyrogenic silica in the presence of hexamethyldisilazane. Silicas thus treated are named “Silica silylate” according to the CTFA (6th edition, 1995). They are, for example, sold under the references “Aerosil R812®” by Degussa and “Cab-O-Sil TS-530®” by Cabot,
dimethylsilyloxyl or polydimethylsiloxane groups, which are obtained in particular by treatment of pyrogenic silica in the presence of polydimethylsiloxane or dimethyldichlorosilane. Silicas thus treated are named “Silica dimethyl silylate” according to the CTFA (6th edition, 1995). They are, for example, sold under the references “Aerosil R972®” and “Aerosil R974®” by Degussa and “Cab-O-Sil TS-610®” and “Cab-O-Sil TS-720®” by Cabot.

The hydrophobic pyrogenic silica exhibits in particular a particle size which can be nanometric to micrometric, for example ranging from approximately 5 to 200 nm.

The polymeric organic lipophilic gelling agents are, for example, partially or completely crosslinked organopolysiloxane elastomers of three-dimensional structure, such as those sold under the names of “KSG6®”, “KSG16®” and “KSG18®” by Shin-Etsu, of “Trefil E-505C®” and “Trefil E-506C®” by Dow Corning, of “Gransil SR-CYC®”, “SR DMF10®”, “SR-DC556®”, “SR 5CYC gel®”, “SR DMF 10 gel®” and “SR DC 556gel®” by Grant Industries, of “SF 1204®” and of “JK 113®” by General Electric; ethylcellulose, such as that sold under the name of “Ethocel®” by Dow Chemical; galactomannans comprising from one to six and in particular from two to four hydroxyl groups per monosaccharide which are substituted by a saturated or unsaturated alkyl chain, such as guar gum alkylated by C₁ to C₆ alkyl chains, in particular C₁ to C₃ alkyl chains, and their mixtures; block copolymers of “diblock” or “triblock” type of the polystyrene/polyisoprene or polystyrene/polybutadiene type, such as those sold under the name “Luvitol HSB®” by BASF, of the polystyrene/copoly(ethylene-propylene) type, such as those sold under the name “Kraton®” by Shell Chemical Co., or of the polystyrene/copoly-(ethylene-butylene) type.

Mention may also be made, among the lipophilic gelling agents which can be used in the compositions according to the invention, of esters of dextrin and of fatty acid, such as dextrin palmitates, in particular such as those sold under the names of “Rheopearl TL®” or “Rheopearl KL®” by Chiba Flour.

The lipophilic gelling agents can be present in the compositions according to the invention in a content ranging from 0.05 to 40% by weight, with respect to the total weight of each composition, preferably from 0.5 to 20% by weight and better still from 1 to 15% by weight.

Mention may be made, as hydrophilic or water-soluble gelling agent, of:
homo- or copolymers of acrylic acid or methacrylic acid or their salts and their esters and in particular the products sold under the names “Versicol F” or “Versicol K” by Allied Colloid, “Ultrahold 8” by Ciba-Geigy, poly(acrylic acid)s of Synthalen K type,
copolymers of acrylic acid and of acrylamide, sold in the form of their sodium salts under the “Reten” names by Hercules, poly(sodium methacrylate), sold under the name “Darvan No. 7” by Vanderbilt, sodium salts of poly(hydroxycarboxylic acid)s, sold under the name “Hydagen F” by Henkel,
copolymers of poly(acrylic acid)s and of alkyl acrylates of Pemulen type,
AMPS (poly(acrylamidomethylpropanesulphonic acid) partially neutralized with aqueous ammonia and highly crosslinked), sold by Clariant,
AMPS/acrylamide copolymers of Sepigel or Simugel type, sold by Seppic, and
copolymers of AMPS and of alkyl methacrylates which are polyoxyethylenated (crosslinked or noncrosslinked), and their mixtures.

Mention may be made, as other examples of water-soluble gelling polymers, of:
proteins, such as proteins of plant origin, such as wheat or soya proteins; proteins of animal origin, such as keratins, for example keratin hydrolysates and sulphonic keratins;
anion, cationic, amphoteric or nonionic chitin or chitosan polymers;
cellulose polymers, such as hydroxyethylcellulose, hydroxypropylcellulose, methylcellulose, ethylhydroxyethylcellulose, carboxymethylcellulose and quaternized cellulose derivatives;
vinyl polymers, such as polyvinylpyrrolidones, copolymers of methyl vinyl ether and of malic anhydride, the copolymer of vinyl acetate and of crotonic acid, copolymers of vinylpyrrolidone and of vinyl acetate, copolymers of vinylpyrrolidone and of caprolactam, or poly(vinyl alcohol);
associative polyurethanes, such as the polymer C₁₆—OE₁₂₀-C₁₆ from Servo Delden (sold under the name SER AD FX1100, molecule comprising a urethane functional group and with a weight-average molecular weight of 1300), OE being an oxyethylene unit, Rheolate 205 comprising a urea functional group, sold by Rheox, or also Rheolate 208 or 204 (these polymers being sold in the pure form) or DW 1206B from Röhm & Haas comprising a C₂₀ alkyl chain and comprising a urethane bond, sold at a solids content in water of 20%. Use may also be made of solutions or dispersions of these associative polyurethanes, in particular in water or in an aqueous/alcoholic medium. Mention may be made, by way of example of such polymers, of SER AD FX1010, SER AD FX1035 and SER AD 1070 from Servo Delden and Rheolate 255, Rheolate 278 and Rheolate 244, sold by Rheox. Use may also be made of the products DW 1206F and DW 1206J, and also Acrysol RM 184 or Acrysol 44, from Röhm & Haas or alternatively Borchigel LW 44 from Borchers,
optionally modified polymers of natural origin, such as:
gums arabic, guar gum, xanthan derivatives or karaya gum;
alginates and carrageenans;
glycoaminoglycans, hyaluronic acid and its derivatives;
shellac resin, gum sandarac, dammars, elemis or copals;
deoxyribonucleic acid;
mucopolysaccharides, such as hyaluronic acid, chondroitin sulphates, and their mixtures.

The hydrophilic gelling agents can be present in the compositions according to the invention in a content ranging from 0.05 to 20% by weight, with respect to the total weight of each composition, preferably from 0.5 to 10% by weight and better still from 0.8 to 5% by weight.

The compositions according to the invention can comprise emulsifying surface-active agents present in particular in a proportion ranging from 0.1 to 30% by weight, with respect to the total weight of each composition, better still from 1 to 15% by weight and better still from 2 to 10% by weight. These surface-active agents can be chosen from anionic, cationic, nonionic, amphoteric or zwitterionic surface-active agents. Reference may be made to the document “Encyclopedia of Chemical Technology, Kirk-Othmer”, volume 22, pp. 333-432, 3rd edition, 1979, Wiley, for the definition of the properties and functions (emulsifying) of surfactants, in particular pp. 347-377 of this reference for the anionic and nonionic surfactants.

The surfactants preferably used in the compositions according to the invention are chosen from:
a) nonionic surface-active agents with an HLB of greater than or equal to 8 to 25° C., used alone or as a mixture; mention may in particular be made of:
oxyethylenated and/or oxypropylenated ethers (which can comprise from 1 to 150 oxyethylene and/or oxypropylene groups) of glycerol;
oxyethylenated and/or oxypropylenated ethers (which can comprise from 1 to 150 oxyethylene and/or oxypropylene groups) of fatty alcohols (in particular of C₈-C₂₄ and preferably C₁₂-C₁₈ alcohols), such as the oxyethylenated ether of cetearyl alcohol comprising 30 oxyethylene groups (CTFA name “Ceteareth-30”) and the oxyethylenated ether of the mixture of C₁₂-C₁₅ fatty alcohols comprising 7 oxyethylene groups (CTFA name “C12-15 Pareth-7”) sold under the name “Neodol 25-7®” by Shell Chemicals;
esters of fatty acid (in particular) of C₈-C₂₄ and preferably C₁₆-C₂₂ acid) and of polyethylene glycol (which can comprise from 1 to 150 ethylene glycol units), such as PEG-50 stearate and PEG-40 monostearate, sold under the name Myrj 52P by ICI Uniquema;
esters of fatty acid (in particular of C₈-C₂₄ and preferably C₁₆-C₂₂ acid) and of the oxyethylenated and/or oxypropylenated glycerol ethers (which can comprise from 1 to 150 oxyethylene and/or oxypropylene groups), such as PEG-200 glyceryl monostearate, sold under the name “Simulsol 220™” by Seppic; polyethoxylated glyceryl stearate comprising 30 ethylene oxide groups, such as the product Tagat S sold by Goldschmidt, polyethoxylated glyceryl oleate comprising 30 ethylene oxide groups, such as the product Tagat O sold by Goldschmidt, polyethoxylated glyceryl cocoate comprising 30 ethylene oxide groups, such as the product Varionic LI 13 sold by Sherex, polyethoxylated glyceryl isostearate comprising 30 ethylene oxide groups, such as the product Tagat L sold by Goldschmidt, and polyethoxylated glyceryl laurate comprising 30 ethylene oxide groups, such as the product Tagat I from Goldschmidt;
esters of fatty acid (in particular of C₈-C₂₄ and preferably C₁₆-C₂₂ acid) and of the oxyethylenated and/or oxypropylenated sorbitol ethers (which can comprise from 1 to 150 oxyethylene and/or oxypropylene groups), such as polysorbate 60, sold under the name “Tween 60” by Uniquema;
dimethicone copolyol, such as that sold under the name “Q2-5220” by Dow Corning;
dimethicone copolyol benzoate (Finsolv SLB 101 and 201 from Fintex);
copolymers of propylene oxide and of ethylene oxide, also known as EO/PO polycondensates, such as, for example, the polyethylene glycol/polypropylene glycol/polyethylene glycol triblock polycondensates sold under the “Synperonic” names, such as “Synperonic PE/L44” and “Synperonic PE/F127” by ICI, and their blends;
and their mixtures.
b) nonionic surface-active agents with an HLB of less than 8 at 25° C., optionally in combination with one or more nonionic surface-active agents with an HLB of greater than 8 at 25° C., such as mentioned above, such as:
esters and ethers of monosaccharides, such as sucrose stearate, sucrose cocoate, sorbitan stearate and their mixtures, such as Arlatone 2121, sold by ICI;
esters of fatty acids (in particular of C₈-C₂₄ and preferably C₁₆-C₂₂ acid) and of polyol, in particular of glycerol or of sorbitol, such as glyceryl stearate, such as the product sold under the name Tegin M by Goldschmidt, glyceryl laurate, such as the product sold under the name Imwitor 312 by Hüls, polyglyceryl-2 stearate, sorbitan tristearate or glyceryl ricinoleate;
the cyclomethicone/dimethicone copolyol mixture sold under the name “Q2-3225C” by Dow Corning;
c) anionic surfactants, such as:
salts of C₁₆-C₃₀ fatty acids, in particular those deriving from amines, such as triethanolamine stearate;
salts of polyoxyethylenated fatty acids, in particular those derived from amines or the alkali metal salts, and their mixtures;
phosphoric esters and their salts, such as “DEA oleth-10 phosphate” (Crodafos N 10N from Croda) or cetyl phosphate (Amphisol K from DSM Nutritional Products);
sulphosuccinates, such as “Disodium PEG-5 citrate lauryl sulphosuccinate” and “Disodium ricinoleamido MEA sulphosuccinate”;
alkyl ether sulphates, such as sodium lauryl ether sulphate;
isethionates;
acylglutamates, such as “Disodium hydrogenated tallow glutamate” (Amisoft HS-21 R, sold by Ajinomoto), and their mixtures.

Use is preferably made of surfactants which make it possible to obtain an oil-in-water or wax-in-water emulsion.

The term “fibre” should be understood as meaning an object with a length L and a diameter D such that L is much greater than D, D being the diameter of the circle in which the cross section of the fibre is framed. In particular, the L/D ratio (or aspect ratio) is chosen within the range from 3.5 to 2500, preferably from 5 to 500 and better still from 5 to 150.

The fibres can in particular be fibres used in the manufacture of textiles and in particular silk, cotton, wool or flax fibres, fibres of cellulose, in particular extracted from wood, Vegetables or algae, of rayon, of polyamide (Nylon®), of viscose, of acetate, in particular of rayon acetate, of poly(p-phenylene terephthalamide) (or of aramid), in particular Kevlar®, of acrylic polymer, in particular of poly(methyl methacrylate) or of poly(2-hydroxyethyl methacrylate), of polyolefin in particular of polyethylene or of polypropylene, of glass, of silica, of carbon, in particular in the graphite form, of polytetrafluoroethylene (such as Teflon®), of insoluble collagen, of polyesters, of poly(vinyl chloride), of poly(vinylidene chloride), of polyvinyl alcohol, of polyacrylonitrile, of chitosan, of polyurethane or of polyethylene phthalate, or fibres formed of a blend of polymers, such as those mentioned above, for example polyamide/polyester fibres.

Of course, a person skilled in the art will take care to choose this or these optional additional compounds and/or their amounts so that the advantageous properties of the corresponding composition according to the invention are not, or not substantially, detrimentally affected by the envisaged addition.

Each of the first, second and optionally additional compositions according to the invention can be provided in particular in the form of a suspension, dispersion, solution, gel, emulsion, in particular oil-in-water (O/W), wax-in-water or water-in-oil (W/O) or multiple (W/O/W or polyol/O/W or O/W/O) emulsion, cream, foam, dispersion of vesicles, in particular of ionic or non-ionic lipids, two-phase or multiphase emulsion, spray, powder or paste, in particular soft paste. Each composition is preferably a leave-in composition.

The method according to the invention can advantageously be used for making up the nails and/or skin and/or lips and/or eyelashes, depending on the nature of the ingredients used. In particular, the first, second and optionally third compositions can be provided, independently, in the form of a solid foundation, lipstick stick or paste, concealer or a product for the outline of the eyes, eyeliner, mascara, eyeshadow, product for making up the body or a product for colouring the skin.

According to one embodiment, the first, second and optionally third compositions are lipstick compositions.

According to another embodiment, the first, second and optionally third compositions are compositions for coating keratinous fibres, such as the eyelashes, eyebrows or nonhead hairs, and more particularly mascaras.

According to another embodiment, the first, second and optionally third compositions are foundation compositions.

A person skilled in the art can chobse the appropriate formulation, and its method of preparation, on the basis of his general knowledge, taking into account, on the one hand, the nature of constituents used, in particular their solubility in the support, and, on the other hand, the application envisaged for each composition.

The invention is illustrated in more detail by the examples described below. Unless otherwise indicated, the amounts shown are expressed as percentage by weight.

### EXAMPLE 1

### Preparation of a Noncrosslinked Polyrotaxane

40.0 g of HOOC-PEG-COOH (1.2×10⁻³ mol, M 33 000) and 160 g of α-cyclodextrin (0.16 mol) are dissolved under hot conditions in 1.35 l of water and then the mixture is cooled to 4° C. during 16 h. The white complex is recovered and freeze dried. 30 g of the dry complex are mixed with 0.34 g of adamantanamine (2.2×10⁻³ mol), 1.0 g of (benzotriazol-1-yloxy)tris(dimethylamino)phosphonium hexafluorophosphate (BOP reagent) (2.3×10⁻³ mol), and 0.40 ml of ethyldiisopropylamine (EDIPA) dissolved in 200 ml of dehydrated DMF. The reaction mixture is left to react at 4° C. for 16 h and then the mixture is washed twice with 400 ml of DMF/methanol (1:1) and twice with 250 ml of methanol by centrifuging.

The product is dissolved in 170 ml of DMSO, precipitated from 800 ml of water and then washed by centrifuging twice with 500 ml of water. After washing, the product is dried under vacuum and 17.6 g of a white powder are obtained.

### EXAMPLE 2

### Preparation of a Noncrosslinked Polyrotaxane

0.9 g of polyethylene glycol-bisamine (abbreviated to PEG-BA), sold by Fluka, and 3.6 g of α-cyclodextrin were dissolved in 30 ml of water at 80° C. and the mixture was maintained at 5° C. overnight, in order to obtain the white paste of the inclusion complex.

The paste was dried, an excess of 2,4-dinitrofluorobenzene (2.4 ml) was added, at the same time as 10 ml of dimethylformamide, and then the mixture was stirred overnight at ambient temperature under a nitrogen atmosphere. The reaction mixture was dissolved in 50 ml of DMSO and precipitated twice from a 0.1% aqueous sodium chloride solution (800 ml) to give a yellow product. The product was collected, washed with water and methanol (three times, respectively) and dried to produce the polyrotaxane (1.25 g).

### EXAMPLE 3

### Preparation of a Polyrotaxane Functionalized by a Crosslinking Agent

10 g of polyrotaxane according to Example 1 were dissolved in 70 ml of a 1N NaOH solution. 0.5 g of a solution of cyanuric chloride in 5 ml of a 1N NaOH solution were subsequently added to the mixture at 5° C. The reaction mixture was reacted at 5° C. for 8 hours and the product is collected by freeze drying.

### EXAMPLE 4

### Mascara

1) First Composition

| A | Water | q.s. for 100 |
|---|---|---|
| | Noncrosslinked polyrotaxanes | 10 |
| | of Example 1 | |
| | Hydroxyethylcellulose | 0.9 |
| | Gum arabic | 3.2 |
| | Preservatives | q.s. |
| B | Potassium cetyl phosphate | 3.2 |
| | (Amphisol K from Givaudan) | |
| | Steareth-2 (Brij 72 from | 0.8 |
| | Uniqema) | |
| | Steareth-20 (Brij 78 from | 2.4 |
| | Uniqema) | |
| | Beeswax | 7 |
| | Carnauba wax | 10 |
| | Paraffin wax | 5 |

Procedure:

The polyrotaxane is placed in the water and then the other constituents of the phase A are added.

The constituents of the phase B (waxes, emulsifiers and premilled pigments) are heated on a water bath with stirring and then the phase A is added with rapid stirring to produce the emulsion.

2) Second Composition

| A | Water | q.s. for 100 |
|---|---|---|
| | Polyethylene oxide- | 3 |
| | tetrasuccinimidyl glutarate 10K | |
| | Hydroxyethylcellulose | 0.9 |
| | Gum arabic | 3.2 |
| | Triethanolamine | 2.4 |
| | Preservatives | q.s. |
| B | Stearic acid | 5.8 |
| | Beeswax | 5 |
| | Carnauba wax | 8 |
| | Black iron oxide | 7 |

The constituents of the phase B (waxes and emulsifiers) are heated on a water bath with stirring and then the phase A is added with rapid stirring to produce the emulsion.

A layer of the first composition is applied to the eyelashes and then a layer of the second composition, comprising the crosslinking agent (polyethylene oxide-tetrasuccinimidyl glutarate), is subsequently applied to the first layer.

A film exhibiting good hold, the pigments then being “trapped” in the matrix of polymers, is obtained on the eyelashes.

### EXAMPLE 5

### Foundation

1) First Composition

| Phase | Chemical name | % by weight |
|---|---|---|
| A | Noncrosslinked polyrotaxanes | 5 |
| | of Example 1 | |
| | Water | 49.6 |
| | Preservatives | 0.75 |
| B | Water | 5 |
| | Glycerol | 5 |
| | Titanium dioxide | 8.01 |
| | Yellow iron oxide | 1.19 |
| | Red iron oxide | 0.57 |
| | Black iron oxide | 0.23 |
| C | Glyceryl stearate (Tegin | 3 |
| | Pellets from Goldschmidt) | |
| | Cetearyl alcohol | 1 |
| | Isononyl isononanoate | 20.5 |
| | Preservatives | 0.15 |
| Total | | 100 |

Procedure:

The polyrotaxane is dissolved in the water and then the other constituents of the phase A are added. The pigments are passed through a triple roll mill (phase B).

The constituents of the phase C are heated on a water bath at 65-70° C. with stirring.

The phase A and the phase B are subsequently mixed, the phase C, still at 65-70° C., is then added with rapid stirring over 10 min, to produce the emulsion, and then cooling is allowed to take place to ambient temperature.

2) Second Composition

| Phase | Chemical name | % by weight |
|---|---|---|
| A | Cyanuric chloride | 0.25 |
| | Sodium hydroxide | 0.16 |
| | Water | q.s. for 100 |
| | Magnesium aluminium silicate | 0.75 |
| | Preservatives | 0.75 |

A layer of the first composition is applied to the skin and then a layer of the second composition, comprising the crosslinking agent (cyanuric chloride), is subsequently applied to the first layer.

A deposited layer on the skin exhibiting good hold, the pigments then being “trapped” in the matrix of polymers, is obtained.

### EXAMPLE 6

### Foundation

1) First Composition

| Phase | Chemical name | % by weight |
|---|---|---|
| A | Noncrosslinked polyrotaxanes | 3 |
| | of Example 1 | |
| | Water | q.s. for 100 |
| | Preservatives | 0.75 |
| B | Water | 5 |
| | Glycerol | 5 |
| | Titanium dioxide | 8.01 |
| | Yellow iron oxide | 1.19 |
| | Red iron oxide | 0.57 |
| | Black iron oxide | 0.23 |
| C | Glyceryl stearate (Tegin | 3 |
| | Pellets from Goldschmidt) | |
| | Cetearyl alcohol | 1 |
| | Isononyl isononanoate | 20.5 |
| | Preservatives | 0.15 |

Procedure:

The polyrotaxane is dissolved in the water and then the other constituents of the phase A are added. The pigments are passed through a triple roll mill (phase B).

The constituents of the phase C are heated on a water bath at 65-70° C. with stirring.

The phase A and the phase B are subsequently mixed, the phase C, still at 65-70° C., is then added with rapid stirring over 10 min, to produce the emulsion, and then cooling is allowed to take place to ambient temperature.

2) Second Composition

| | | |
|---|---|---|
| Phase | Chemical name | % by weight |

| | |
|---|---|
| Noncrosslinked polyrotaxanes | 2 |
| of Example 3 | |
| Water | q.s. for 100 |
| Magnesium aluminium silicate | 0.75 |
| Preservatives | 0.75 |

The polyrotaxane is dissolved in the water at ambient temperature and the other ingredients are added.

A layer of the first composition is applied to the skin and then a layer of the second composition, comprising the crosslinking agent (cyanuric chloride), is subsequently applied to the first layer. A deposited layer on the skin exhibiting good hold is obtained.

### EXAMPLE 7

### Foundation

The following composition, which comprises noncrosslinked polyrotaxanes functionalized with a crosslinking agent, is prepared.

| Phase | Chemical name | % by weight |
|---|---|---|
| A | Cyclopentasiloxane | 25 |
| | Isododecane | 6 |
| | Cetyl dimethicone copolyol | 2.7 |
| | (Abil EM 90 from Goldschmidt) | |
| | Polyglyceryl-4 isostearate | 0.9 |
| | (Isolan GI 34 from Goldschmidt) | |
| B | Cyclopentasiloxane | 5 |
| | Coated titanium dioxide | 8.01 |
| | Coated yellow iron oxide | 1.19 |
| | Coated red iron oxide | 0.57 |
| | Coated black iron oxide | 0.23 |
| C | Polyrotaxanes of Example 3 | 5 |
| | Water | q.s. for 100 |
| | Glycerol | 5 |
| | Magnesium sulphate | 0.7 |
| | Preservatives | 0.7 |

Procedure:

The polyrotaxane is dissolved in water at ambient temperature and then the other constituents of the phase C are added.

The constituents of the phase A are mixed at a temperature not exceeding 25° C. with stirring and then the pigments, passed beforehand through the triple roll mill, are added (phase B), and also the phase C, with rapid stirring, to produce the emulsion while remaining at 25° C.

When the composition is applied to the skin, the polyrotaxanes crosslink at the temperature of the skin, which makes it possible to obtain a film possessing good hold.

### EXAMPLE 8

### Foundation

The first composition and the second composition of Example 5 above can be mixed at the time of use in proportions of 50/50 and then at least one layer of the said mixture can be applied to the skin.

1. Cosmetic method for making up or for the nontherapeutic care of keratinous substances chosen from the nails, skin, lips or eyelashes, the method consisting in:
a. depositing on the said keratinous substances at least one layer of at least one first composition comprising at least one first noncrosslinked polyrotaxane and at least one second noncrosslinked polyrotaxane,
b. subjecting the said composition, simultaneously with or subsequent to its application, to at least one chemical, physicochemical and/or mechanical stimulus.

2. Method according to claim 1, characterized in that the stimulus is of thermal, photochemiexal, chemical and/or mechanical nature.

3. Method according to claim 1 or 2, characterized in that the stimulus comprises at least one crosslinking agent.

4. Cosmetic method for making up or for the nontherapeutic care of keratinous substances chosen from the nails, skin, lips or eyelashes, the method consisting in depositing, on the said keratinous substances:
a. at least one layer of a first composition comprising at least one first noncrosslinked polyrotaxane,
b. at least one layer of a second composition comprising a crosslinking agent,
the first composition and/or the second composition comprising at least one second noncrosslinked polyrotaxane.

5. Cosmetic method for making up or for the nontherapeutic care of keratinous substances chosen from the nails, skin, lips or eyelashes, the method consisting in depositing, on the said keratinous substances, at least one layer of a first composition comprising at least one first noncrosslinked polyrotaxane and at least one second noncrosslinked polyrotaxane, the first noncrosslinked polyrotaxane and/or the second noncrosslinked polyrotaxane being grafted with a crosslinking agent.

6. Cosmetic method for making up or for the nontherapeutic care of keratinous substances chosen from the nails, skin, lips or eyelashes, the method consisting in depositing, on the said keratinous substances:
at least one layer of a first composition comprising at least one first noncrosslinked polyrotaxane,
at least one layer of a second composition comprising at least one second noncrosslinked polyrotaxane,
at least one of the first or second noncrosslinked polyrotaxanes being grafted with a crosslinking agent.

7. Cosmetic method for making up or for the nontherapeutic care of keratinous substances chosen from the nails, skin, lips and eyelashes, consisting in:
a. mixing, at the time of use:
at least one first composition comprising a cosmetically acceptable medium and at least one first noncrosslinked polyrotaxane, and
at least one second composition comprising a crosslinking agent,
the first composition or the second composition comprising at least one second noncrosslinked polyrotaxane, then
b. applying at least one layer of the said mixture to the nails, skin, lips or eyelashes.

8. Method according to one of claims 1 to 7, characterized in that the first and the second noncrosslinked polyrotaxanes each comprise at least one linear molecule and at least two cyclic molecules.

9. Method according to claim 8, characterized in that the linear molecule of the first polyrotaxane and the linear molecule of the second polyrotaxane are chosen, independently of one another, from:
hydrophilic polymers, such as a poly(vinyl alcohol), a polyvinylpyrrolidone, a poly((meth)acrylic acid), polymers derived from cellulose (carboxymethylcellulose, hydroxyethylcellulose, hydroxypropylcellulose and the like), a polyacrylamide, polyalkylene glycols, such as polyethylene glycols and polypropylene glycols, polytetrahydrofurans, poly(vinyl acetal)s, a poly(vinyl methyl ether), polyamines, polyethyleneimine, casein, gelatin, starch, and their copolymers;
hydrophobic polymers, for example polyolefins, such as polyethylenes, polypropylenes, polyisoprenes, polyisobutylenes or polybutadienes; copolymers of olefins, such as ethylene/butylene copolymers; polyesters, polydimethylsiloxanes, poly(vinyl chloride), polystyrene, acrylonitrile/styrene copolymers, polymers and copolymers of (meth)acrylic esters, such as poly(methyl methacrylate) or acrylonitrile/methyl acrylate copolymers; polycarbonates, polyurethanes, vinyl chloride/vinyl acetate copolymers or poly(vinyl butyral);
and their derivatives.

10. Method according to either of claims 8 and 9, characterized in that the linear molecule of the first polyrotaxane and the linear molecule of the second polyrotaxane are chosen, independently of one another, from polyethylene glycols and polypropylene glycols.

11. Method according to any one of claims 8 to 10, characterized in that the linear molecule of the first polyrotaxane and the linear molecule of the second polyrotaxane exhibit a weight-average molecular weight of greater than or equal to 350 g/mol, for example ranging from 350 to 2 000 000, preferably ranging from 1500 to 1 000 000, more preferably from 2800 to 800 000, better still from 7000 to 700 000, for example ranging from 10 000 to 600 000 or from 10 000 to 500 000.

12. Method according to any one of claims 8 to 11, characterized in that the cyclic molecules of the first and second polyrotaxanes are chosen, independently of one another, from:
cyclodextrins, for example a-cyclodextrin, β-cyclodextrin, γ-cyclodextrin, dimethylcyclodextrin and glucosylcyclodextrin, and their derivatives,
crown ethers,
benzocrown ethers, dibenzocrown ethers and dicyclohexanocrown ethers, and their derivatives.

13. Method according to claim 12, characterized in that the cyclic molecules are α-cyclodextrins.

14. Method according to any one of claims 8 to 13, characterized in that the ratio of the number of cyclic molecules strung along the linear molecule of each polyrotaxane to the maximum amount of cyclic molecules of the same nature which could be strung along the linear molecule ranges from 0.01 to 0.6, preferably from 0.01 to 0.5 and better still from 0.05 to 0.4.

15. Method according to any one of claims 8 to 14, characterized in that the linear molecule of the first polyrotaxane and the linear molecule of the second polyrotaxane comprise, at each of their ends, independently of one another, a blocking molecular structure, the molecular structure being a molecule or a macromolecule.

16. Method according to claim 15, characterized in that the molecular structure carries an ionic charge and/or occupies a volume such that it prevents the cyclic molecules and the linear molecule from separating.

17. Method according to either one of claims 15 and 16, characterized in that the molecular structure is chosen from
dinitrophenyl groups, such as the 2,4- and 3,5-dinitrophenyl groups;
cyclodextrins;
adamantane groups;
trityl groups;
fluoresceins;
pyrenes;
naphthalimides; and
their combinations.

18. Method according to one of claims 3 to 17, characterized in that the crosslinking agent is chosen from compounds having at least two polymerizable double bonds, compounds having at least one polymerizable double bond and at least one functional group which reacts with the noncrosslinked polyrotaxane or polyrotaxanes, compounds having at least two functional groups which react with the noncrosslinked polyrotaxane or polyrotaxanes, cyanuric chloride, trimesoyl chloride, terephthaloyl chloride, epichlorohydrin, dibromobenzene, glutaraldehyde, bis(acid chlorides), tri(acid chlorides) and the like, polyvalent metallic compounds which form ionic crosslinkings, coupling agents of silane type and/or titanium-based coupling agents, photocrosslinking agents and their mixtures.

19. Method according to the preceding claim, characterized in that the crosslinking agent is chosen from cyanuric chloride, tolylene 2,4-diisocyanate, 1,1′-carbonyldiimidazole, trimesoyl chloride, terephthaloyl chloride, alkoxysilanes, such as tetramethoxysilane and tetraethoxysilane, cycloaliphatic epoxides, such as 3,4-epoxycyclohexylmethyl 3,4-epoxycyclohexanecarboxylate, polyethylene oxide-succinimidyl glutarates, such as polyethylene oxide-tetrasuccinimidyl glutarate, bishydrazides, and the like, and their mixtures.

20. Method according to one of the preceding claims, characterized in that the first and second noncrosslinked polyrotaxanes are present in a content ranging from 0.1 to 80% by weight, preferably from 1 to 50% by weight and more preferably from 2 to 30% by weight, with respect to the total weight of each first or second composition.

21. Method according to one of the preceding claims, characterized in that the first and/or second composition comprises a liquid fatty phase comprising at least one oil or organic solvent chosen from volatile oils, non-volatile oils and their mixtures.

22. Method according to the preceding claim, characterized in that the liquid fatty phase comprises at least one volatile hydrocarbon oil having from 8 to 16 carbon atoms.

23. Method according to claim 21 or 22, characterized in that the oil or oils represent from 1% to 80% by weight, preferably from 5% to 50% by weight, with respect to the total weight of the first and/or the second composition.

24. Method according to one of the preceding claims, characterized in that the first and/or the second composition comprises an aqueous phase.

25. Method according to the preceding claim, characterized in that the aqueous phase represents from 5 to 95% by weight, with respect to the total weight of the composition comprising it, preferably from 10 to 85% by weight.

26. Method according to any one of the preceding claims, characterized in that the first and/or the second composition comprises at least one fatty substance which is solid or pasty at ambient temperature chosen from waxes, pasty compounds and their mixtures.

27. Method according to any one of the preceding claims, characterized in that the first and/or the second composition comprises from 0.1 to 70% by weight of waxes, with respect to the total weight of the composition, better still from 1 to 60% by weight and even better still from 5 to 40% by weight.

28. Method according to any one of the preceding claims, characterized in that the first and/or the second composition comprises at least one film-forming polymer.

29. Method according to claim 28, characterized in that the film-forming polymer is present in a content of dry matter ranging from 0.1% to 30% by weight, with respect to the total weight of the composition, preferably from 0.5% to 20% by weight and better still from 1% to 15% by weight.

30. Method according to one of the preceding claims, characterized in that the first and/or the second composition comprises a cosmetic ingredient chosen from vitamins, thickeners, lipophilic or hydrophilic gelling agents, trace elements, softeners, sequestering agents, fragrances, basifying or acidifying agents, preservatives, sunscreens, antioxidants, fibres, care agents or their mixtures.

31. Kit for making up or for the nontherapeutic care of keratinous substances chosen from the nails, skin, lips or eyelashes, comprising:
i) at least one first composition comprising at least one first noncrosslinked polyrotaxane,
ii) at least one second composition comprising a crosslinking agent,
the first composition and/or the second composition comprising at least one second noncrosslinked polyrotaxane.

32. Kit according to claim 31, characterized in that the first and the second compositions are packaged separately in the same article of packaging.
